(19) United States
(12) Patent Application Publication
(10) Pub. No.: US 2026/0185983 A1
(43) Pub. Date: Jul. 2, 2026

(54) METHODS FOR MONITORING AND FOR ASSESSING THE EFFICACY OF TREG CELL THERAPIES

(71) Applicant: PolTREG S.A., Gdansk (PL)

(72) Inventors: Maciej ZIELINSKI, Gdansk (PL); Justyna SAKOWSKA, Gdansk (PL); Piotr TRZONKOWSKI, Gdansk (PL)

(21) Appl. No.: 18/722,923

(22) PCT Filed: Sep. 11, 2023
(86) PCT No.: PCT/EP2023/074939

§ 371 Date: Jun. 21, 2024

(51) Int. Cl. G01N 33/50; C07K 16/28; G01N 33/569

(52) U.S. Cl. CPC G01N 33/5091; C07K 16/2887; G01N 33/56972

(57) ABSTRACT

The present invention relates to methods for monitoring and for assessing the efficacy of, respectfully, cell therapies of patients treated with CD4+ Fox P3+ T regulatory cells. Further subject matter relates to cell therapeutic methods using CD4+ Fox P3+ T regulatory cells and the monitoring methods of the invention.

The present invention relates to methods for monitoring and for assessing the efficacy of, respectfully, cell therapies of patients treated with CD4+ Fox P3+ T regulatory cells (in the following Treg cells” or “Tregs”). Further subject matter relates to cell therapeutic methods using CD4+ Fox P3+ T regulatory cells and the monitoring methods of the invention.

Both clinical and laboratory data have proved the importance of the immune system in the pathogenesis of type 1 diabetes mellitus (DM1). While the triggering factor may be a viral infection or genetic susceptibility, the disease develops due to an imbalance between exaggerated auto-aggressive T cells responses and impaired tolerogenic mechanisms (1, 2). The most promising disease-modifying strategies are therefore created around immunotherapy (3,4). Only recently, teplizumab was introduced, the first-in-century drug approved by the FDA for the treatment of pre-symptomatic DM1 individuals. It is a monoclonal antibody targeting autoreactive CD3+ T cells, which allows for at least two years delay in the onset of symptomatic DM1 (5). Among many ongoing attempts to stop or at least delay DM1, cell therapy with FoxP3+ T regulatory cells (Treg) seems to be of particular interest. The present inventors and others performed several clinical trials with T regulatory cells product with promising results (5-10).

The major challenge for all these therapies is an excellent immune marker to predict the treatment response, which correlates with β-cell destruction leading to the DM1 (11). The tissue available for sampling is usually peripheral blood, which is very distant from local tissue lesions. Thus, the function of β-cells remains the only acceptable endpoint of therapeutic efficacy so far (4). Unfortunately, this sort of monitoring allows us to detect the disease relatively late when the destruction of the islets is ongoing. It shows only the progression of DM1 and does not allow predicting the efficacy of the treatment early, at the time of the therapy administration.

The technical problem underlying the present invention is the provision of means for evaluating the progress of cell therapies using Treg cells.

The solution to the to the above technical problem is provided by the embodiments of the present invention as characterized in the claims and disclosed in the present description and the accompanying drawings.

The present invention is based on the finding that, as set out herein and in further detail in detail in the Example below, certain biomarkers are useful for characterizing cell therapeutic regimen in patients in need of such cell therapy using Treg cells. The biomarkers identified as particular useful for this purpose are PD-1 and CD73. Moreover, the inventors have determined that certain values of the expression of PD-1 and/or CD73 indicated an efficient cell therapy treatment with Treg cells.

In particular, the present invention provides a method for monitoring a cell therapy of a patient treated with CD4+ Fox P3+ Treg cells comprising the step(s) of determining the expression of at least one protein selected from the group consisting of PD-1 and CD73 on CD4+ FoxP3+ T cells, preferably CD4+ FoxP3+ Treg cells, and/or determining the expression of PD-1 on CD4+ FoxP3− T cells, preferably CD4+ FoxP3− T effector cells (“Teff cells” or “Teffs”), of the patient in vitro.

Preferably, determining the expression of the at least one protein on CD4+ FoxP3+ T cells and/or determining the expression PD-1 on CD4+ FoP3− cells is carried out on (in other instances of the present disclosure synonymously used as “carried out with”) a sample of the patient.

In a preferred embodiment, the monitoring method of the invention comprises, before determining the expression of the at least one protein, isolating CD4+ FoxP3+ T cells from a sample of the patient and/or, before determining the expression of PCD1 on CD4+ FoxP3− cells, isolating CD4+ FoxP3− cells from the sample of the patient. Isolation of the cells of interest according to the invention as disclosed herein, in particular from peripheral blood, is typically carried out according to methods known in the art which typically comprises the isolation of peripheral blood mononuclear cells (PBMCs), and a specific method is described in more detail in the Example below.

More preferably, the isolated CD4+ FoxP3+ T cells and/or the isolated CD4+ FoxP3− cells are expanded before determining the expression of the biomarkers according to the invention as outlined above. The term “expanding isolated cells” as used herein means that said cells, after their isolation from a patient's sample, are cultured under appropriate conditions and for an appropriate time known in the art and as exemplified in the Example below, for allowing to increase the number of the cells to be analysed in a given culture or a test sample taken therefrom to perform the detection of the expression of the protein of interest on said cells.

Preferably, the sample of the patient is peripheral blood.

As outlined above, the isolated and, optionally, expanded CD4+ FoxP3+ T cells are preferably CD4+ FoxP3+ T regulatory cells.

Furthermore, the isolated and, optionally, expanded CD4+ FoxP3− T cells are preferably CD4+ FoxP3− T effector cells.

In preferred embodiments of the invention the monitoring method comprises the steps of:
(i) determining the expression of PD-1 on the CD4+ FoxP3+ T regulatory cells; and/or
(ii) determining the expression of PD-1 on the CD4+ FoxP3− T effector cells; and/or
(iii) determining the expression of CD73 on the CD4+ FoxP3+ T regulatory cells.

In certain embodiments of the invention the above monitoring method preferably comprises the above step (i). In other embodiments of the invention the above monitoring method preferably comprises the above step (ii). In further embodiments of the invention the above monitoring method preferably comprises the above step (iii). In still other embodiments of the invention the above monitoring method preferably comprises the above steps (i) and (ii). Yet in other embodiments of the invention the above monitoring method preferably comprises the above steps (i) and (iii). In further embodiments of the invention the above monitoring method preferably comprises the above steps (ii) and (iii). In still further embodiments of the invention the above monitoring method preferably comprises the above steps (i), (ii) and (iii).

In case two or more of the above steps (i), (ii) and (iii) are carried out, the steps can be carried out simultaneously or sequentially.

In preferred embodiments, T regulatory cells used in the invention are of the phenotype CD4+ FoxP3+ CD25high CD127− doublets−.

In further preferred embodiments, T effector cells used in the invention are of the phenotype CD4+ FoxP3− CD25low CD127+ doublets−.

The method according to any one of the preceding claims wherein the method is carried out on the cells of the patient at least once, preferably at least 2 weeks, preferably 1 month, more preferably 2 months, even more preferred 3 months after the administration of the cell therapy.

In other embodiments of the invention, the method of the invention is carried out on said cells (CD4+ FoxP3, preferably CD4+ FoxP3+ CD25high CD127− doublets−, and/or CD4+ FoxP3− CD25low CD127+ doublets−, T cells) more than once. More preferably the method of the invention is carried out in intervals which intervals may be regular or irregular, preferably regular. It is particularly preferred that the monitoring method is carried out in a continuous fashion for continuously monitoring the cell therapy of the patient, preferably the monitoring method of the invention is carried out as long as the cell therapeutic treatment of the patient continues. The method can be carried out until a certain endpoint is reached such as, for example, until a pre-determined threshold of expression of PD-1 and/or CD73 on the CD4+ FoxP3+, preferably CD4+ FoxP3+ CD25high CD127− doublets− (Treg) cells and/or a predetermined threshold of expression of PD-1 on the preferably CD4+ FoxP3− CD25high CD127− doublets− (Teff), cells is reached.

“Threshold of expression” in the context of the invention is preferably a certain percentage value of the respective cells expressing the respective biomarker for use in the invention based on the total number of those cells present in the determination, preferably in a test sample, more preferably in a test sample obtained from the patient, still more preferred in a test sample after isolation from the sample of the patient, most preferred in a test sample after expansion, of said isolated cells. Such a predetermined threshold may be a value of the expression of the respective biomarker for use in the invention on the respective cells being a maximum value or a minimum value.

A maximum or minimum, respectively, value as an endpoint threshold may be a pre-determined absolute maximum value or minimum value over the complete duration of the cell therapy. In other embodiments, the maximum or minimum, respectively, value may be a maximum or minimum value within a pre-determined time window of duration of the cell therapy such as about 1 to 6 months, such as about 1, about 2, about 3, about 4, about 5, or about 6 months, preferably a within a time window of about 1 year, about 1.5 years or about 2 years.

One preferred threshold value according to the invention is the expression of PD-1 on at least, more preferably more than, 16% of the CD4+ FoxP3+ T cells, preferably Treg cells, based on the total number of said cells in a test sample, such as a volume of a test sample containing a certain number of CD4+ FoxP3+ T cells, preferably Treg cells. Another preferred threshold value according to the invention is the expression of CD73 on at least, more preferably more than, 7% of the CD4+ FoxP3+ T cells, preferably Treg cells, based on the total number of said cells in a test sample, such as a volume of a test sample containing a certain number of CD4+ FoxP3+ T cells, preferably Treg cells. A further preferred threshold value according to the invention is the expression of PD-1 on at least, more preferably more than, 8% of the CD4+ FoxP3− T cells, preferably Teff cells, based on the total number of said cells in a test sample, such as a volume of a test sample containing a certain number of CD4+ FoxP3− T cells, preferably Teff, cells. In certain embodiments of the invention at least one of the above preferred threshold values of the above biomarkers on the respective cells determines an endpoint. In other embodiments of the invention at least two of the above threshold values determines an endpoint. In one embodiment the endpoint is determined by the above preferred threshold value of PD-1 on said of CD4+ FoxP3+ T cells, preferably Treg cells, together with above threshold value of CD73 on said of CD4+ FoxP3+ T cells, preferably Treg cells. In other embodiments the endpoint is determined by the above preferred threshold value of PD-1 on said of CD4+ FoxP3+ T cells, preferably Treg cells, together with above threshold value of PD-1 on said CD4+ FoxP3− T cells, preferably Teff cells. In a further embodiment the endpoint is determined by the above preferred threshold value of CD73 on said CD4+ FoxP3+ T cells, preferably Treg cells, together with above threshold value of PD-1 on said CD4+ FoxP3− T cells, preferably Teff cells. In a further embodiment, the endpoint is determined by all three of the above preferred threshold values. In preferred embodiments, the preferred endpoints as outlined before are reached when the expression of said at least one, or said at least two, or said three, biomarkers is/are below the value(s) indicated above whereby it is understood that, during the monitoring method, the expression value of said biomarker(s) has at least reached once said threshold value(s) before the expression value has reached the respective endpoint value(s).

Preferably the method is carried out on the cells of the patient in intervals of at least about 2 weeks, preferably at least about 1 month, more preferably at least about 2 months, even more preferred at least about 3 months after onset of the cell therapy, i.e. after the administration of the CD4+ FoxP3+ Treg cells. “Onset” of the cell therapy is defined herein as day zero, i.e. the first administration of the CD4+ FoxP3+ Treg cells (in other instances of the present disclosure it is also referred to as “first administration of the cell therapy”)

In preferred embodiments of the invention the monitoring method is carried out on the cells of the patient for at least about 6 months, more preferably at least about 1 year, more preferably at least about 2 years, after the administration, preferably the first administration of the cell therapy, in particular administration of the CD4+ FoxP3+ Treg cells, preferably CD4+ FoxP3+, preferably CD4+ FoxP3+ CD25high CD127− doublets− (Treg) cells, whereby the monitoring method is preferably carried in the above-described intervals.

It is further preferred that the monitoring method is carried out at least once on said cells of the patient prior to the administration of the cell therapy, in particular at least once prior to administration of the CD4+ FoxP3+ Treg cells, preferably CD4+ FoxP3+, preferably CD4+ FoxP3+ CD25high CD127− doublets− (Treg) cells. That is, the monitoring method of the invention comprises the determination of base line value(s) of expression on the above-described respective cells before the onset of the cell therapy.

The determination of said expression value(s) of the biomarkers for use in the invention on respective cells, as outlined above, may be carried out in various ways known in the art such as western blot, detection of mRNA of the interested proteins cell sorting and the like.

Preferred methodology for said determination(s), e.g. western blot, cell sorting etc., is the treatment of a suitable test sample with compounds, preferably antibodies, more preferably, monoclonal antibodies which also include suitable antibody fragments specific for the respective biomarker(s). It is further preferred to use cell sorting, more preferably fluorescence-activated cell sorting (FACS), for determining the percentage of the respective cells expressing the respective biomarkers. Specific determination steps which may be used for carrying out the invention are further outlined in detail in the Example below.

The monitoring method of the invention is particularly useful for monitoring a cell therapy using the respective Treg cells in patients having or developing, respectively, an autoimmune disease. Autoimmune diseases in the context of the invention may be one or more of the following:
Acromegaly;
Acquired aplastic anemia;
Acquired hemophilia;
Agammaglobulinemia, primary;
Alopecia areata;
Ankylosing spondylitis (AS);
Anti-NMDA receptor encephalitis;
Antiphospholipid syndrome (APS)| catastrophic antiphospholipid syndrome (CAPS)/Asherson's syndrome;
Arteriosclerosis;
Autoimmune Addison's disease (AAD);
Autoimmune autonomic ganglionopathy (AAG)/autoimmune dysautonomia|autoimmune gastrointestinal dysmotility (AGID);
Autoimmune encephalitis|acute disseminated encephalomyelitis (ADEM);
Autoimmune gastritis;
Autoimmune hemolytic anemia (AIHA);
Autoimmune hepatitis (AIH);
Autoimmune hyperlipidemia;
Autoimmune hypophysitis;
Autoimmune inner ear disease (AIED);
Autoimmune lymphoproliferative syndrome (ALPS);
Autoimmune myelofibrosis;
Autoimmune myocarditis;
Autoimmune oophoritis;
Autoimmune pancreatitis (AIP);
Autoimmune polyglandular syndromes, types I, II, & III (APS type 1, APS type 2, APS type 3, APECED);
Autoimmune progesterone dermatitis;
Autoimmune retinopathy (AIR);
Autoimmune sudden sensorineural hearing loss (SNHL);
Balo disease;
Behçet's disease;
Birdshot chorioretinopathy/birdshot uveitis;
Bullous pemphigoid;
Castleman disease;
Celiac disease;
Chagas disease;
Chronic inflammatory demyelinating polyneuropathy (CIDP);
Chronic autoimmune urticaria;
Churg-Strauss syndrome/eosinophilic granulomatosis with polyangiitis (EGPA);
Cogan's syndrome;
Cold agglutinin disease;
CREST syndrome|limited cutaneous systemic sclerosis;
Crohn's disease (CD);
Cronkhite-Canada syndrome (CSS);
Cryptogenic organizing pneumonia (COP);
Dermatitis herpetiformis;
Dermatomyositis;
Type 1 diabetes mellitus (DM1)
Discoid lupus;
Dressler's syndrome/postmyocardial infarction/postpericardiotomy syndrome;
Eczema/Atopic Dermatitis;
Endometriosis;
Eosinophilic esophagitis;
Eosinophilic fasciitis;
Erythema nodosum;
Essential mixed cryoglobulinemia;
Evans syndrome;
Fibrosing alveolitis/Idiopathic pulmonary fibrosis (IPF);
Giant cell arteritis/temporal arteritis/Horton's disease;
Giant Cell Myocarditis;
Glomerulonephritis;
Goodpasture's syndrome/anti-GBM/anti-TBM disease;
Granulomatosis with polyangiitis (GPA)/Wegener's granulomatosis;
Graves disease/thyroid eye disease;
Guillain-Barré syndrome (GBS);
Hashimoto's thyroiditis/chronic lymphocytic thyroiditis/autoimmune thyroiditis;
Henoch-Schönlein purpura/IgA vasculitis;
Hidradenitis suppurativa;
Hurst's disease/acute hemorrhagic leukoencephalitis (AHLE);
Hypogammaglobulinemia;
IgA nephropathy/Berger's disease;
Immune-mediated necrotizing myopathy (IMNM);
Immune thrombocytopenia (ITP)/autoimmune thrombocytopenic purpura/autoimmune thrombocytopenia;
Inclusion body myositis;
IgG4-related sclerosing disease (ISD);
Interstitial cystitis;
Juvenile idiopathic arthritis/Adult-onset Still's disease;
Juvenile polymyositis|Juvenile dermatomyositis|juvenile myositis;
Kawasaki disease;
Lambert-Eaton myasthenic syndrome (LEMS);
Leukocytoclastic vasculitis;
Lichen planus;
Lichen sclerosus;
Ligneous conjunctivitis;
Linear IgA disease (LAD)|linear IgA bullous dermatosis (LABD);
Lupus nephritis;
Lyme disease/chronic Lyme disease/post-treatment Lyme disease syndrome (PTLDS);
Lymphocytic colitis/microscopic colitis;
Lymphocytic hypophystitis/autoimmune hypophystitis;
Ménière's disease;
Microscopic polyangiitis (MPA)/ANCA-associated vasculitis;
Mixed connective tissue disease (MCTD);
Mooren's ulcer;
Mucha-Habermann disease;
Multifocal motor neuropathy;
Multiple sclerosis (MS);
Myalgic encephalomyelitis (ME)/Chronic fatigue syndrome (CFS);
Myasthenia gravis (MG);
Narcolepsy;
Neuromyelitis Optica/Devic's disease;
Ocular cicatricial pemphigoid;
Opsoclonus-myoclonus syndrome (OMS);
Palindromic rheumatism;
Paraneoplastic cerebellar degeneration;
Paraneoplastic Pemphigus;
Parry-Romberg syndrome (PRS)/Hemifacial atrophy (HFA)/Progressive facial hemiatrophy;
Paroxysmal nocturnal hemoglobinuria (PNH);
Peripheral uveitis/pars planitis;
PANS/PANDAS;
Parsonage-Turner syndrome;
Pemphigus gestationis/Herpes gestationis;
Pemphigus foliaceus;
Pemphigus vulgaris;
Pernicious anemia;
POEMS syndrome;
Polyarteritis nodosa;
Polymyalgia rheumatica;
Polymyositis;
Postural orthostatic tachycardia syndrome (POTS);
Primary biliary cirrhosis (PBC)/primary biliary cholangitis;
Primary sclerosing cholangitis (PSC);
Psoriasis;
Palmoplantar Pustulosis;
Psoriatic arthritis;
Pulmonary fibrosis, idiopathic (IPF);
Pure red cell aplasia (PRCA);
Pyoderma gangrenosum;
Rasmussen's encephalitis;
Raynaud's syndrome/phenomenon;
Reactive arthritis/Reiter's syndrome;
Reflex sympathetic dystrophy syndrome (RSD)/Complex regional pain syndrome (CRPS);
Relapsing polychondritis;
Restless leg syndrome (RLS)/Willis-Ekbom disease;
Rheumatic fever;
Rheumatoid arthritis;
Sarcoidosis;
Schmidt syndrome/autoimmune polyendocrine syndrome type II;
Scleritis;
Scleroderma;
Sclerosing Mesenteritis/Mesenteric Panniculitis;
Serpiginous choroidopathy;
Sjogren's syndrome;
Stiff person syndrome (SPS);
Small fiber sensory neuropathy;
Systemic lupus erythematosus (SLE);
Subacute bacterial endocarditis (SBE);
Subacute cutaneous lupus;
Susac syndrome;
Sydenham's chorea;
Sympathetic ophthalmia;
Takayasu's arteritis (vasculitis);
Testicular autoimmunity (vasculitis, orchitis);
Tolosa-Hunt syndrome;
Transverse myelitis (TM);
Tubulointerstitial nephritis uveitis syndrome (TINU);
Ulcerative colitis (UC);
Undifferentiated connective tissue disease (UCTD);
Uveitis|anterior/intermediate/posterior;
Vasculitis;
VEXAS Syndrome;
Vitiligo and
Vogt-Koyanagi-Harada syndrome (VKH)

Preferably, the autoimmune disease is type 1 diabetes mellitus (DM1).

In preferred embodiments of the invention, the patient is a child or an adolescent, in particular a child or an adolescent having or developing, respectively, DM1. Preferably, the patient having or developing, respectively, an autoimmune disease, preferably DM1, is of an age of about 7 to about 18 years, more preferably about 8 to about 16 years. In further preferred embodiments of the invention, with respect to all of the monitoring, diagnostic, assessment and treatment methods defined and disclosed herein, in particular concerning an autoimmune disease, particularly DM-1, the patient, preferably a child or adolescent, preferably of an age as outlined above, is in an early stage or early onset of the autoimmune disease, preferably DM1. In preferred embodiments of the invention, “early stage” or “early onset”, respectively, of the disease, especially in the context of DM-1, means that the patient has a fasting state plasma C-peptide level of more than about 0.7 ng/ml and/or, preferably and, increase in plasma C-peptide level in the GST (glucagon-stimulated c-peptide test (42); 1 mg glucagon i.v. within 10 seconds) by at least 100% compared to the plasma c-peptide level in the fasting state. In other preferred embodiments of the invention, “early stage” or “early onset”, respectively, of the disease, especially in the context of DM-1, means that the patient has a fasting state plasma C-peptide level of more than about 0.7 ng/ml and/or, preferably and, increase in plasma C-peptide level in a mixed meal tolerance test (MMT), preferably as disclosed in (41), by at least 100% compared to the plasma c-peptide level in the fasting state.

A further aspect of the invention is a method for the assessment of the efficacy of a cell therapy of a patient treated with CD4+ Fox P3+ T regulatory cells comprising carrying out the monitoring method of the present invention wherein the expression of PD-1 on at least, more preferably more than, 16% of the CD4+ FoxP3+ T regulatory cells and/or the expression of CD73 on at least, more preferably more than, 7% of the CD4+ FoxP3+ T regulatory cells and/or the expression of PD-1 on at least, more preferably more than, 8% of the CD4+ FoxP3− T cells indicates an efficient cell therapy. It is understood that the percentage of said cells is based on the total number of said cells in a test sample as outlined above with respect to preferred threshold or endpoint, respectively values in the context of the monitoring method of the present invention.

The cell therapy may also comprise the administration of further medication useful in the treatment of the respective disease, preferably an autoimmune disease such as those outlined above, most preferred DM1.

In the context of a cell therapy as outlined above, it is preferred that the cell therapy is carried out under the further administration of at least one anti-CD20 antibody or fragment thereof having anti-CD20 affinity, i.e., a fragment of said antibody retaining specific binding to CD20. Preferably, the anti-CD20 antibody is a monoclonal antibody, more preferably the anti-CD20 monoclonal antibody is a humanized antibody. Most preferred, the anti-CD20 antibody is rituximab (in the following also denoted as “RTX”). The administration of the Treg cells and the anti-CD20 antibody or fragment thereof, most preferred RTX, can be simultaneously or non-simultaneously. Preferably, administration of the anti-CD20 antibody or fragment thereof, most preferred, entails an amount of about 200 mg/m² of body surface area (“BSA”) to about 400 mg/m² of body surface, more preferred about 330 mg/m² BSA to about 390 mg/m² BSA, most preferred about 375 mg/m² BSA of the patient. Preferably, the administration of a unit dose of the anti-CD20 antibody or fragment thereof, most preferred RTX, is carried out more than once wherein the administration is preferably by intravenous administration. The administration of the anti-CD20 antibody or fragment thereof, preferably RTX, is preferably carried out in intervals which may be regular or non-regular. Preferred regimens are administration of a unit dose, preferably as outlined above, are intervals of about one to about two weeks such as intervals about 6 to 15 days such as 7, 8, 9, 10, 11, 12, 13, or 14 days.

The BSA value of the patient may be calculated form body weight and height, and, optionally, age of the patient. In preferred embodiments of the invention the BSA is calculated according to a formula known in the art such as the Boyd formula, Dubois formula, Gehan-George formula, Haycock formula, Mosteller formula or Takahira formula. In the context of treatment of a child or adolescent according to the invention the calculation of the BSA is preferably carried out according to the Gehan-George formula and/or the Haycock formula.

Antibodies for use in the invention can be polyclonal or monoclonal. Preferred antibodies for use in the invention are monoclonal antibodies.

According to the invention, a “fragment of an antibody having affinity” and/or “a fragment retaining specific binding” to the respective antigen (such as CD20, PD-1 and/or CD73) refers to one or more fragments of an antibody retaining the ability to specifically bind to the respective antigen. Examples such antibody fragments include a Fab fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, an alternative scaffold protein, and a fusion protein thereof.

The term “specific binding” as used herein preferably means that the respective entity such an antibody or fragment thereof as outlined above shows a dissociation constant (K_{D}) for its binding to the target, typically the antigen of an antibody such as antibodies and fragments thereof, disclosed herein for use in the invention, being in the range of less than about 10⁻⁶ M or less, preferably about 10⁻⁷ M or less, more preferably about 10⁻⁸ M or less, even more preferred about 10⁻⁹ M or less, most preferred about 10⁻¹⁰ M or less.

Further subject matter of the present invention is a cell therapy method of a patient in need thereof, preferably a patient such as a child or an adolescent, preferably having or developing, respectively, an autoimmune disease such as an autoimmune disease as disclosed above, most preferred DM1, comprising the administration of Treg cells as outlined above, and carrying out the monitoring and/or assessment method of the invention, preferably further comprising the administration of rituximab. Preferably, the cell therapy method is carried out until at least one threshold value and/or endpoint is reached as detailed above.

The present invention is also directed to the use of Treg cells as described herein for treatment of a patient, preferably a patient such as a child or an adolescent, preferably having or developing, respectively, an autoimmune disease such as an autoimmune disease as disclosed above, most preferred DM1, by cell therapy comprising the administration of said Treg cells, and optionally an anti-CD20-antibody or fragment thereof as defined herein, most preferred rituximab, and further comprising carrying out the assessment method and/or the monitoring method of the invention. Preferably, the cell therapy of said patient is carried out until at least one threshold value and/or endpoint is reached as detailed above. In preferred embodiments, the dose of Treg cells for use in the treatment as defined herein, in particular CD4+ FoxP3+ Treg cells, most preferred CD4+ FoxP3+ CD25high CD127− doublets− Treg cells, is preferably as outlined below.

Preferred dosages of Treg cells in the context of the invention are unit doses in the range of from about 10×10⁶ cells to about 90×10⁶, preferably about 20×10⁶ to about 70×10⁶, more preferably about 30×10⁶ to about 60×10⁶, such as about 30×10⁶, about 40×10⁶, about 50×10⁶, or about 60×10⁶, most preferred about 30×106 Treg cells per kg body weight of the patient which are typically administered in one dose per administration, preferably intravenous (i.v.) administration.

A unit dose of the Treg cells for use in the invention, preferably a unit dose as described above, comprises the Treg cells in a suitable medium, preferably adapted to i.v. administration such as physiological NaCl solution, in an amount of preferably about 100 to about 500 ml, preferably about 200 to about 300 ml, such as about 250 ml medium such as 0.9% (w/v) NaCl in water for injection.

The administration of the Treg cells is carried out at least once. Preferably, the administration is carried out more than once wherein the interval between administrations may be equal or may be different. Preferred intervals in the context of the treatments according to the present invention are about 1 to about 6 months, preferably about 2 to 4 months, most preferred about 3 months. In certain preferred embodiments of the invention administration of Treg cells is carried out, preferably using in a unit dose as outlined above, at least 2 times within an interval as outlined before, most preferred 2 or more times in an interval of 3 months.

According to the invention, it is understood that the term “treatment of a patient having a disease” such as an autoimmune disease, preferably an autoimmune disease as outlined above, most preferred DM1, entails the treatment of said disease in said patient, preferably a child or an adolescent. The term “treatment” as used herein comprises a delay in the development of a disease such as those as outlined herein, most preferred DM-1, such as a delay of at least about 6 months, preferably at least about 1 year, more preferably at least about 1.5 years, still further preferred at least about 2 years or more such as at least about 3 years, at least about 4 years, at east about 5 years, or at least about 6 years or more. As used herein “delay in the development” of a disease” such as an autoimmune disease as disclosed herein, preferably DM-1, preferably means that the condition of the patient is such that time until which the patient develops the clinical stage of the disease according to general knowledge of and the accepted clinical standards by the skilled person, in particular a physician, is prolonged, preferably by a time period as outlined above. In the context of DM1, the delay in the progress or development respectively preferably means that the time until the patient has developed an accepted clinical stage where an insulin therapy of the patient becomes necessary according to the accepted clinical standards is prolonged, preferably for a time period as outlined above.

Further subject matter of the invention is a method of diagnosing an efficient cell therapy of a patient treated with CD4+ Fox P3+ T regulatory cells comprising the steps of obtaining a sample of a patient, isolating CD4+ FoxP3+ T cells and/or CD4+ FoxP3− cells from the patient, contacting the isolated CD4+ FoxP3+ T cells and/or isolated CD4+ FoxP3− cells with at least one detectable compound having affinity for (i.e. specifically binding to, preferably showing a K_{D} as outlined above) PD-1 and/or CD73, detecting said compound bound to CD4+ FoxP3+ T cells expressing PD-1 and/or CD73 and/or bound to CD4+ FoxP3− cells expressing PD-1, respectively, and determining the proportion of the CD4+ FoxP3+ T cells expressing PD-1 and/or CD73 and/or the proportion of CD4+ FoxP3− cells expressing PD-1, preferably in a test sample.

The invention is also directed to the use of said detectable compound in the diagnostic method according to the invention as defined in the previous paragraph.

Preferably, the isolated CD4+ FoxP3+ T cells and/or isolated CD4+ FoxP3− cells are expanded before contacting said cells with said detectable compound.

Preferably, the detectable compound is an anti-PD1-antibody or fragment thereof retaining specific binding to PD-1 or an anti-CD73-antibody or fragment thereof retaining specific binding to CD73.

It is further preferred that the detectable compound such as an anti-PD-1 antibody or an anti-CD73 antibody (or a fragment of such antibody as defined herein) for is coupled to a detectable label, preferably a fluorescent label. Preferred fluorescent labels according to the invention include, but are not limited to, the fluorochromes as shown in Table 3A.

In the above defined diagnostic method comprising obtaining a sample of the patient, the sample is preferably peripheral blood. With respect to preferred embodiments and techniques, respectively, for isolation of the cells, expansion of the cells and detection of bound detectable compound and determination of the proportion of the cells bound to the detectable compound it is referred to the above-described in vitro monitoring method.

Preferably, the proportion of cells bound to the respective detectable compound indicating an efficient cell therapy is as outlined above.

The Figures show:

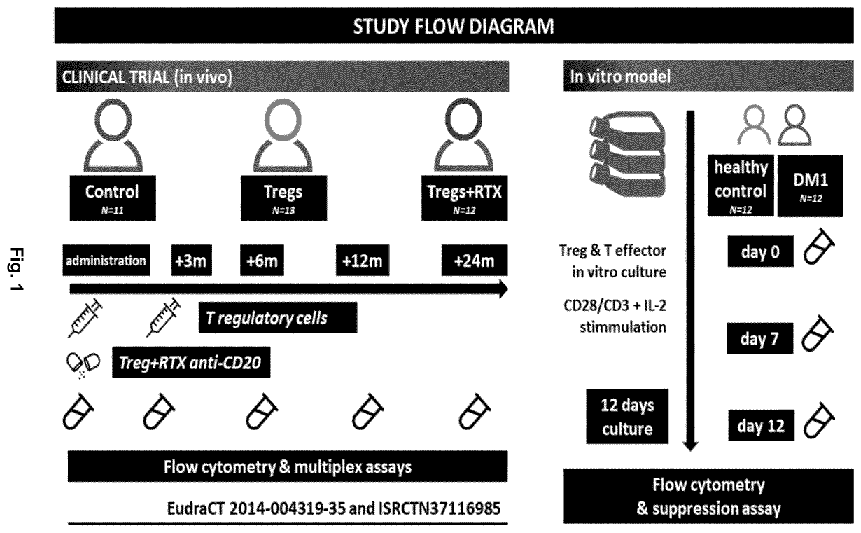

FIG. 1 Study flow diagram for the clinical trial and in vitro model experiment according to the Example of the present invention.

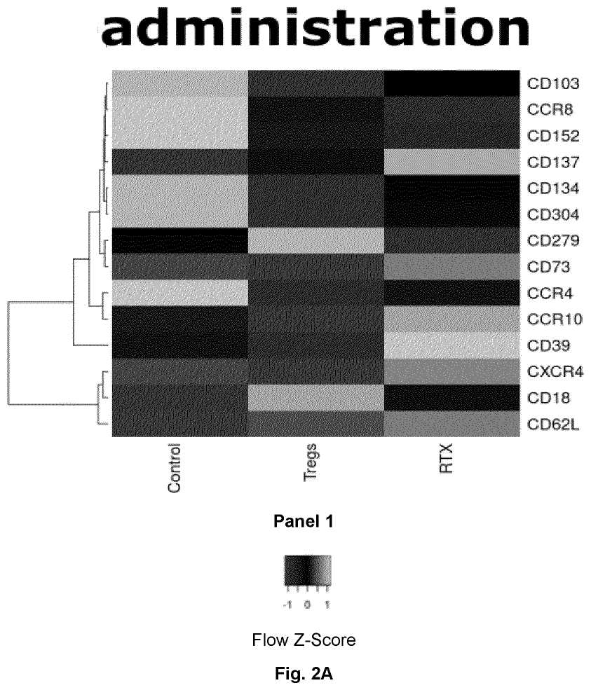

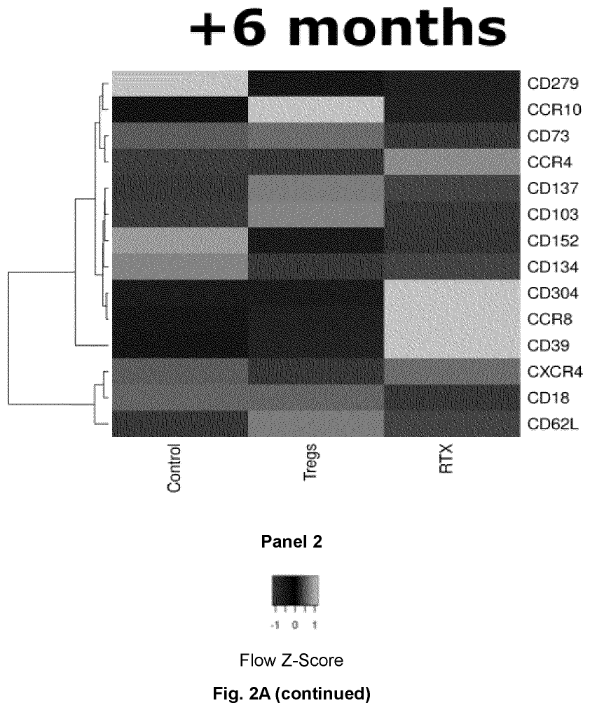

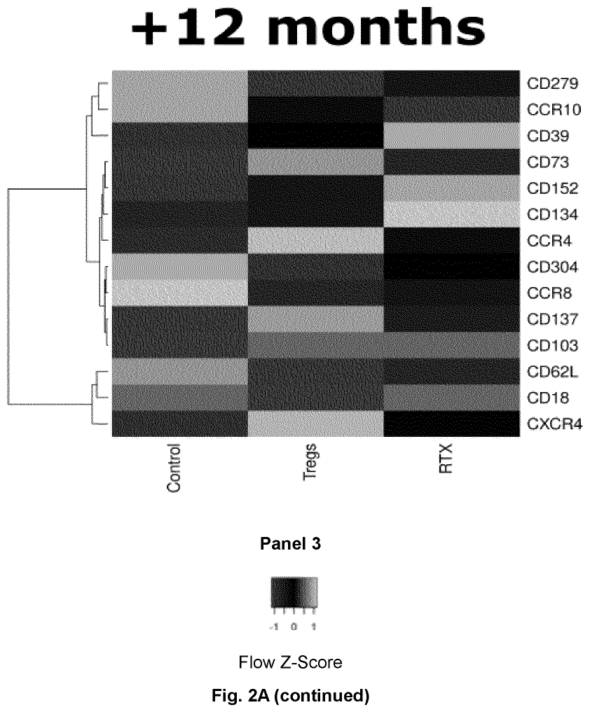

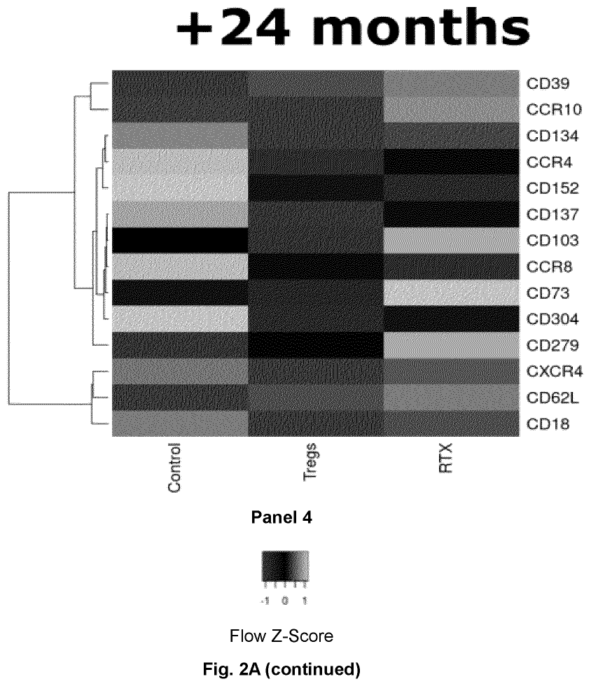

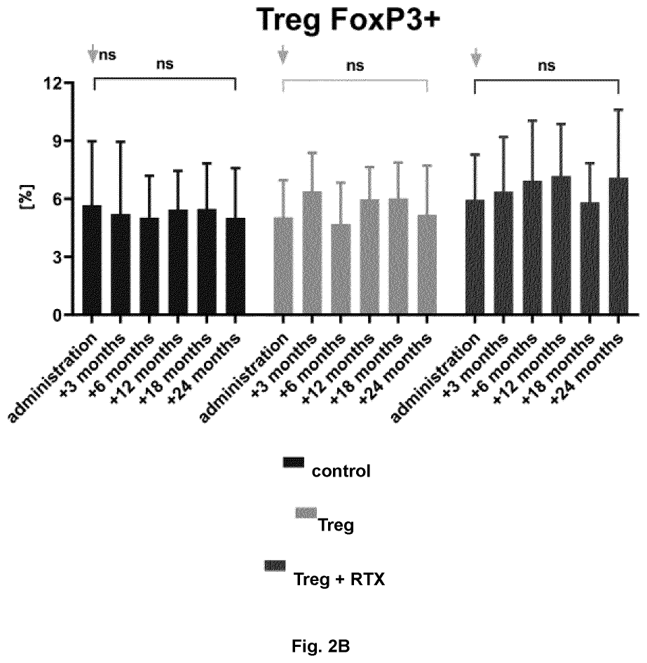

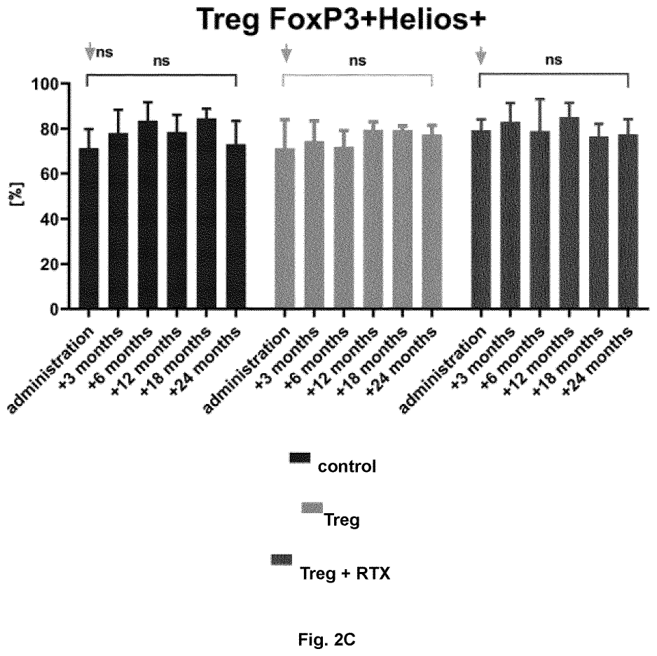

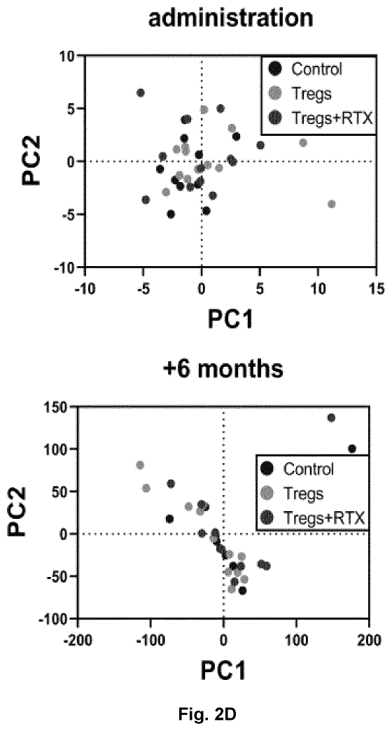

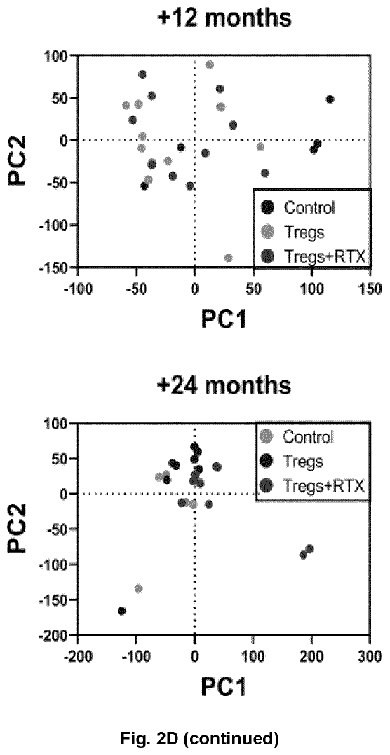

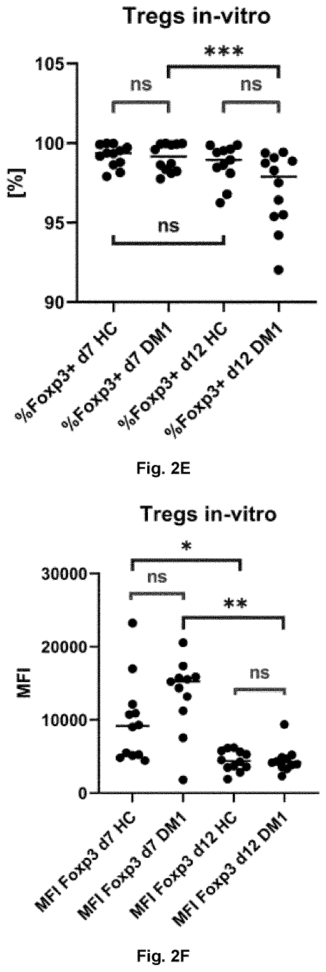

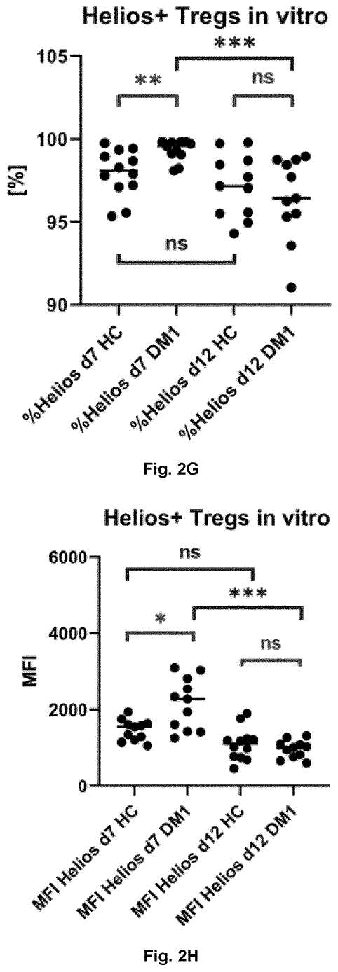

FIG. 2 Regulatory T cells phenotype during patient follow-up, compared to the in vitro model.

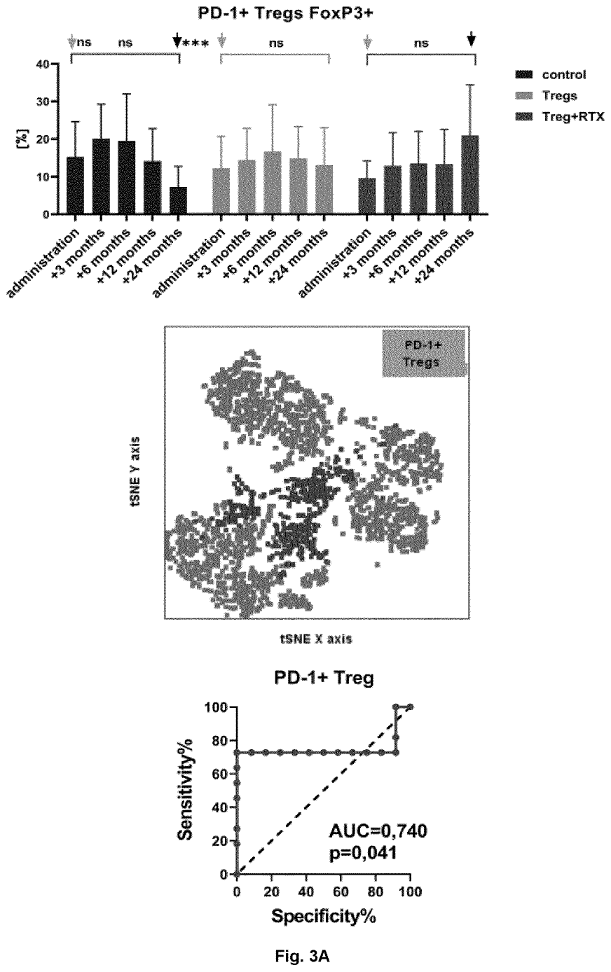

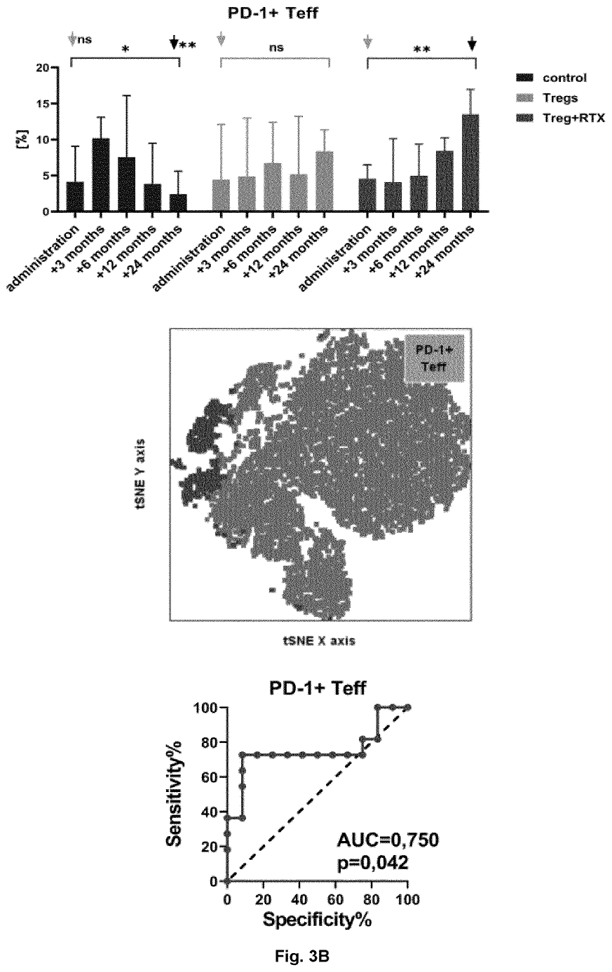

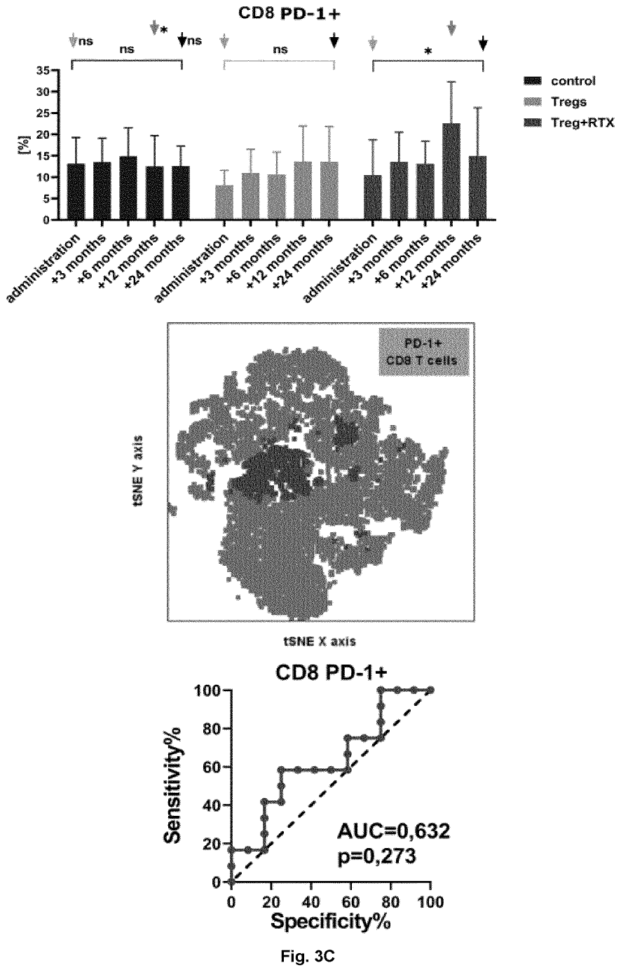

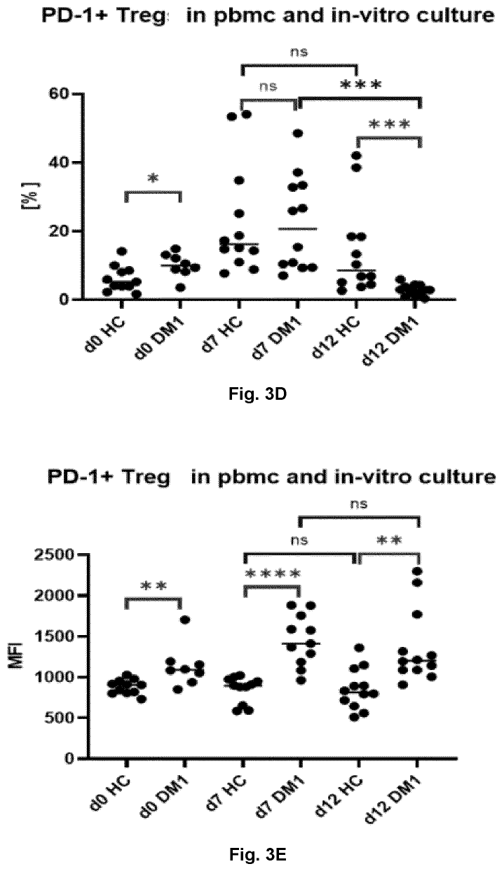

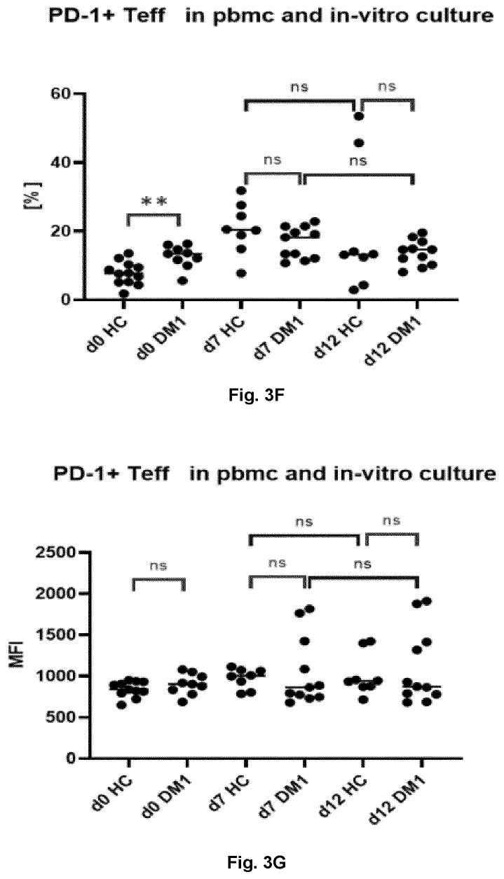

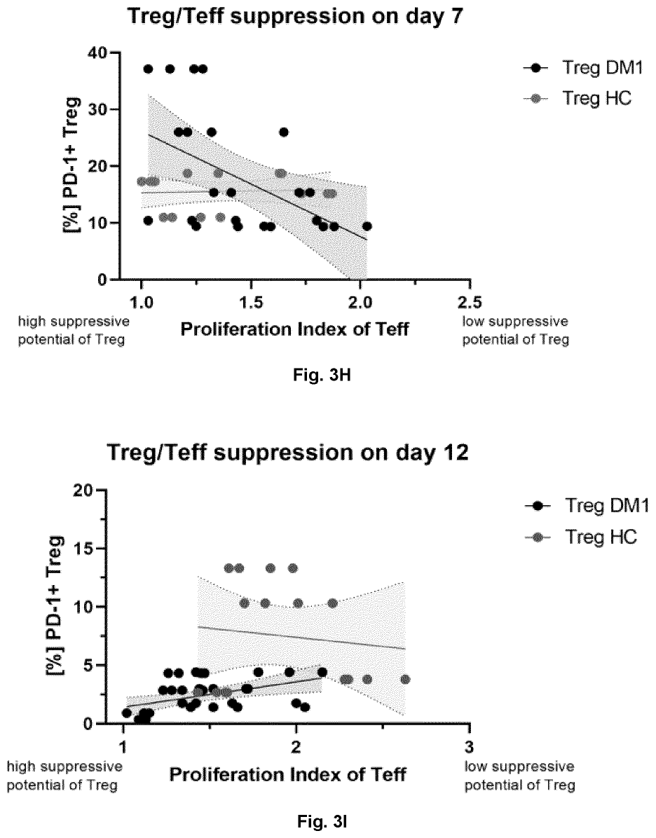

FIG. 3 Expression of PD-1 on regulatory T cells and effector T cells during patient follow-up, compared to the in vitro model.

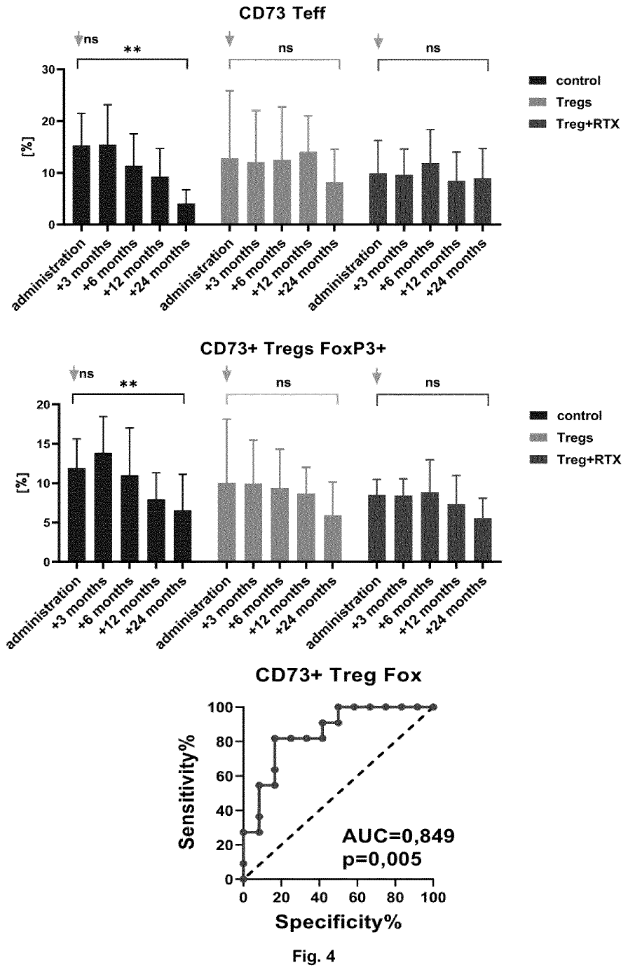

FIG. 4 Surface expression of CD73 on regulatory T cells (Tregs FoxP3+) and effector CD4+ T cells from administration up to 2 years follow-up. The ROC curve for the percentage of CD73+ Tregs in patients treated with polyclonal regulatory T cells and rituximab.

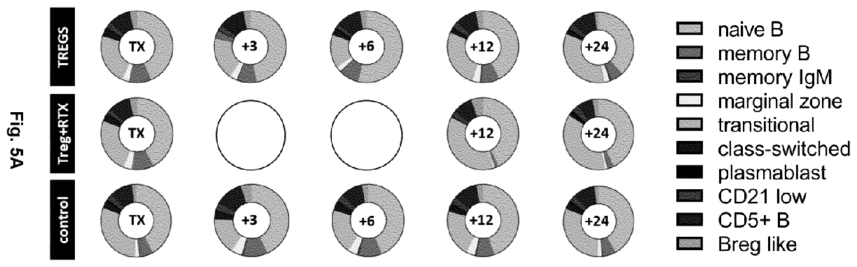

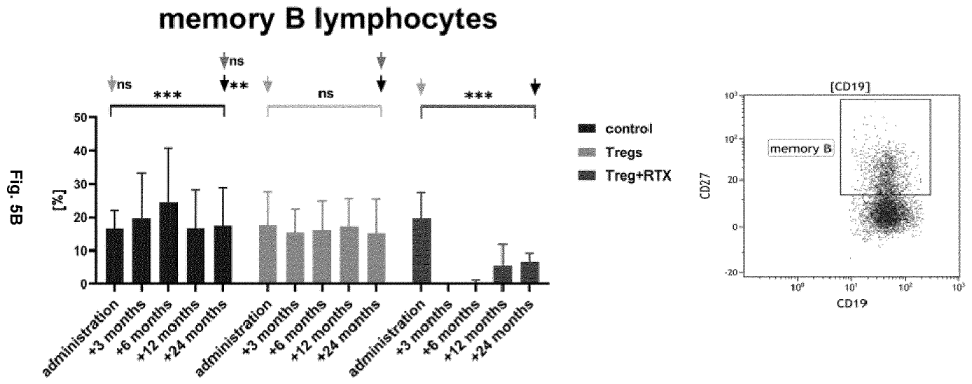

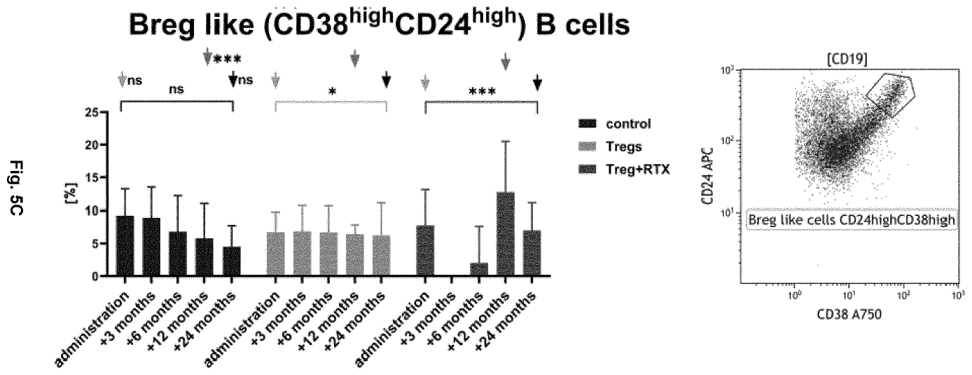

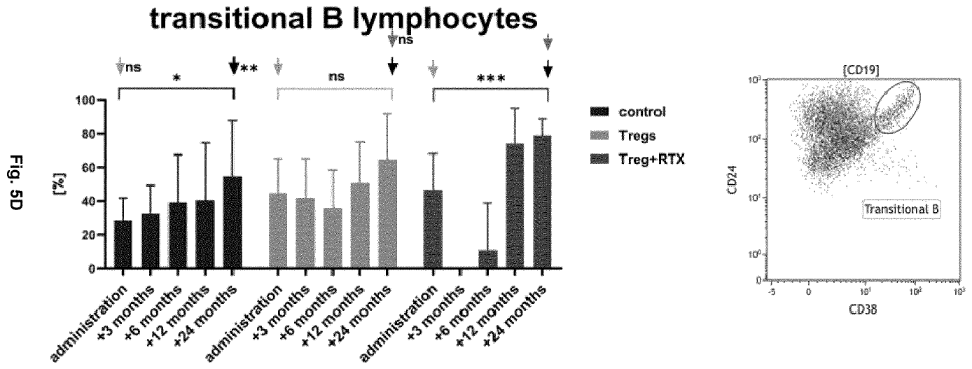

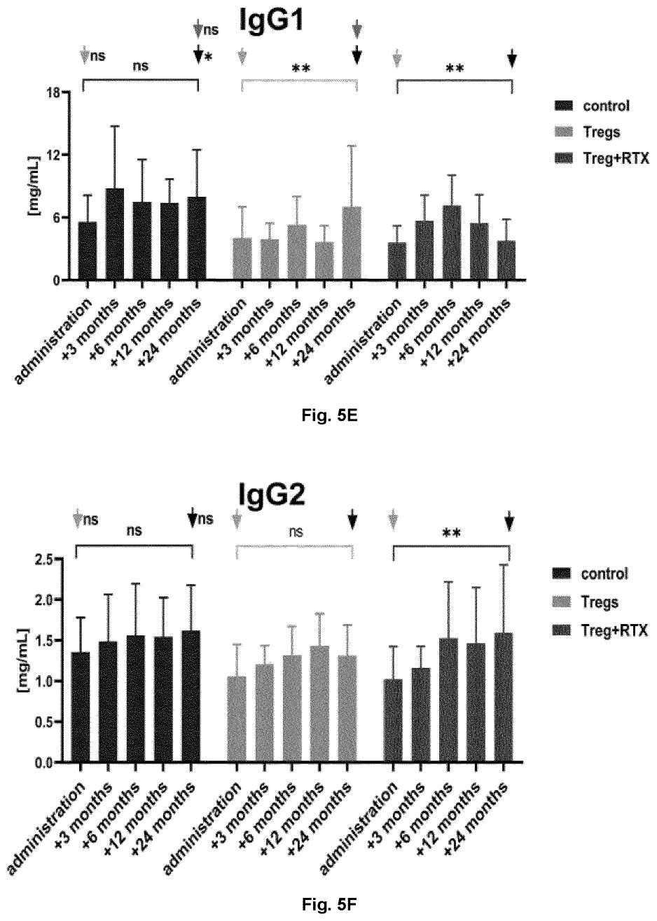

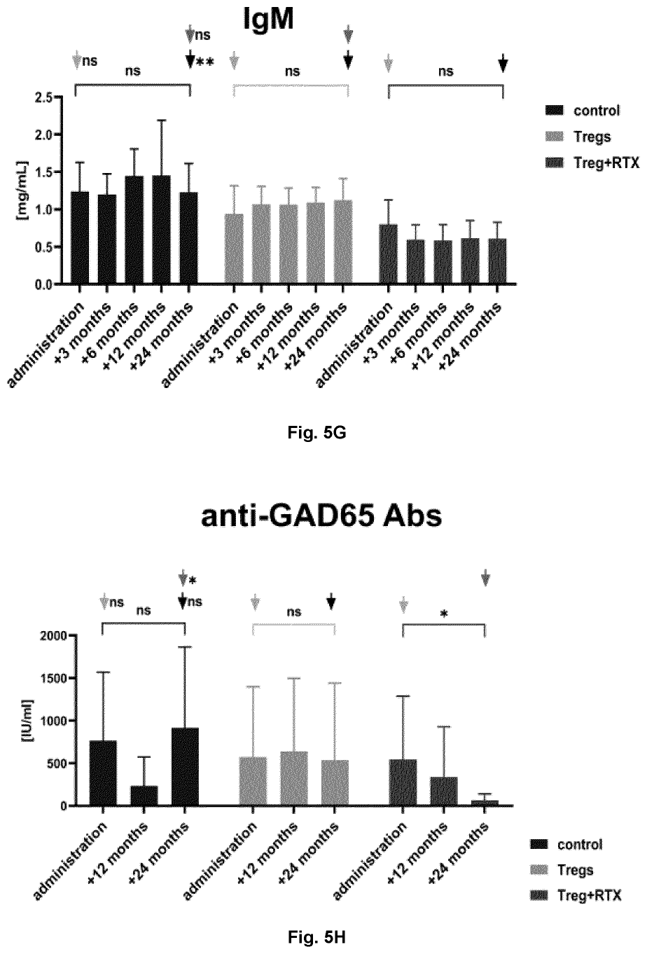

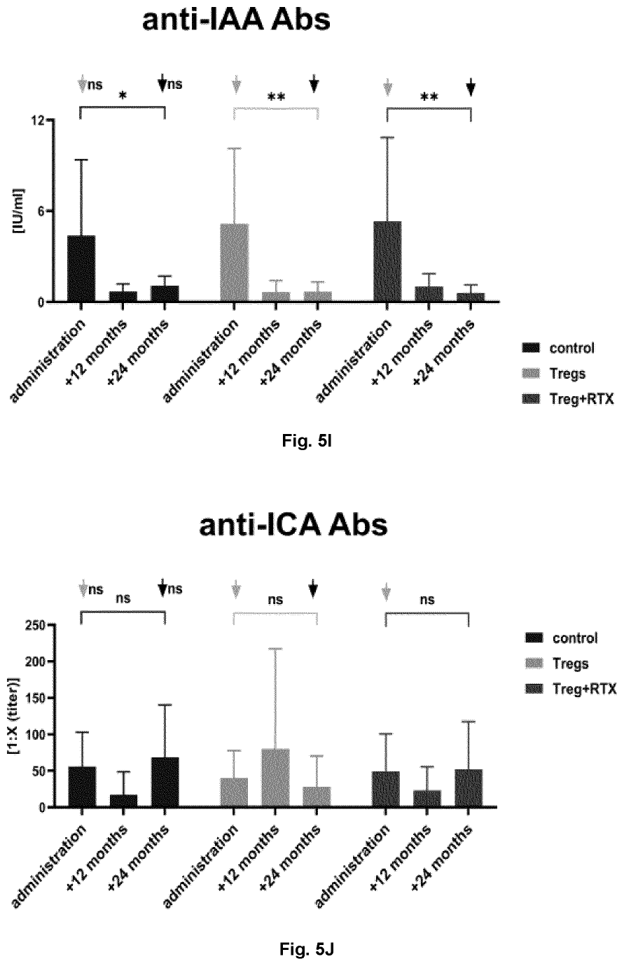

FIG. 5 Evolution of patients' humoral immune response up to two years post-therapy.

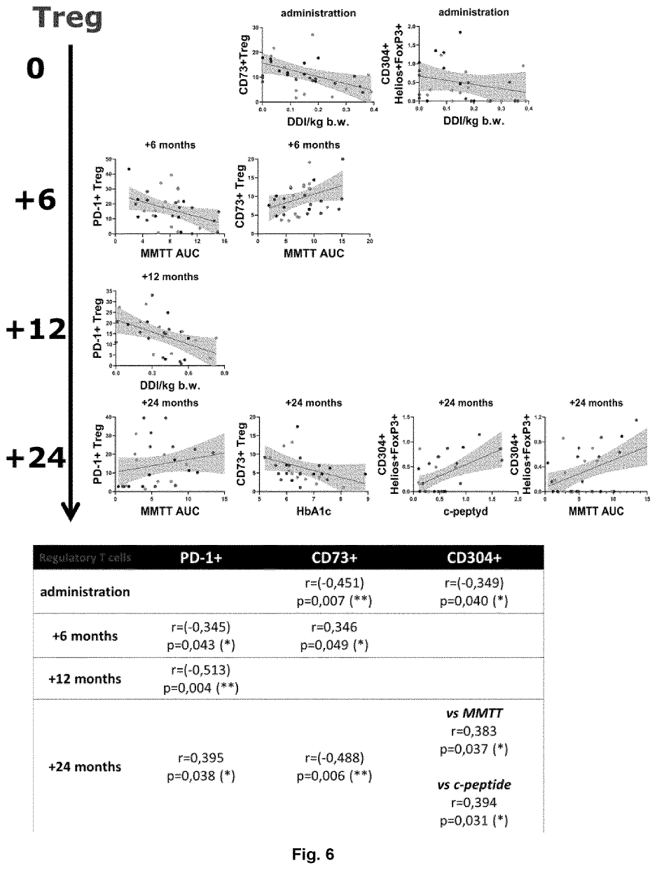

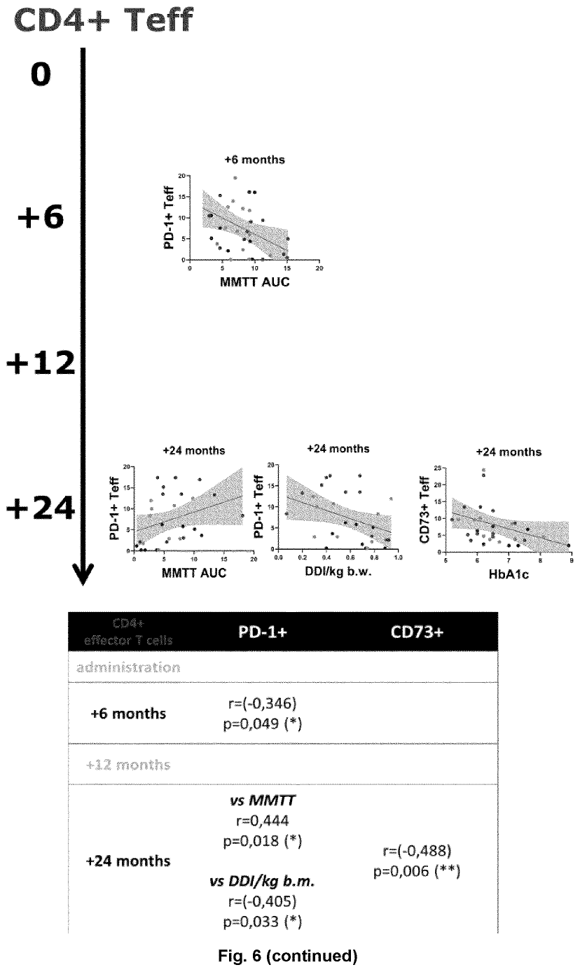

FIG. 6: Clinical correlates of regulatory T cells and effector T cells during patient's follow-up.

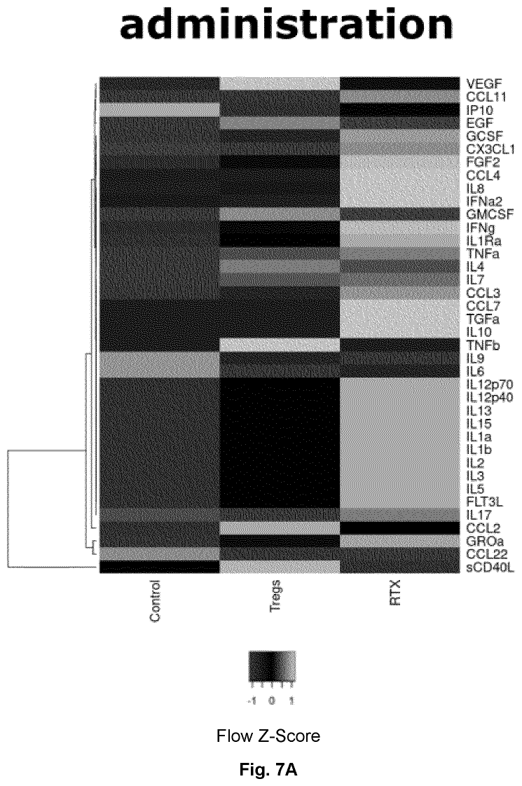

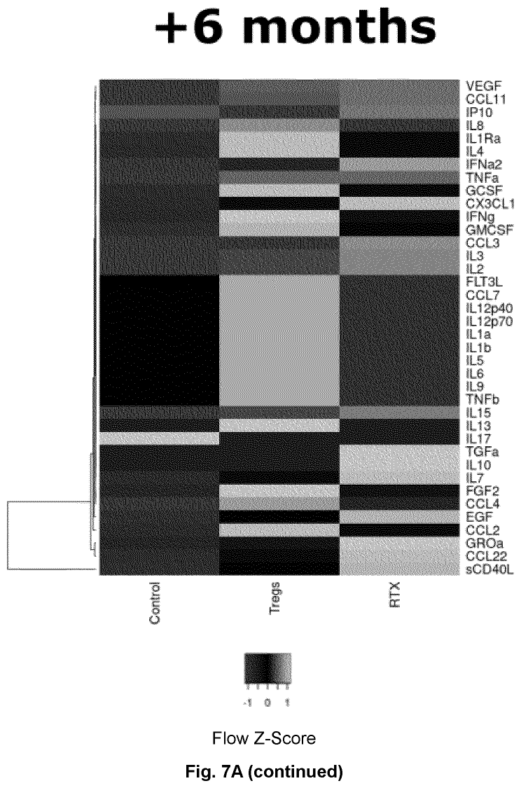

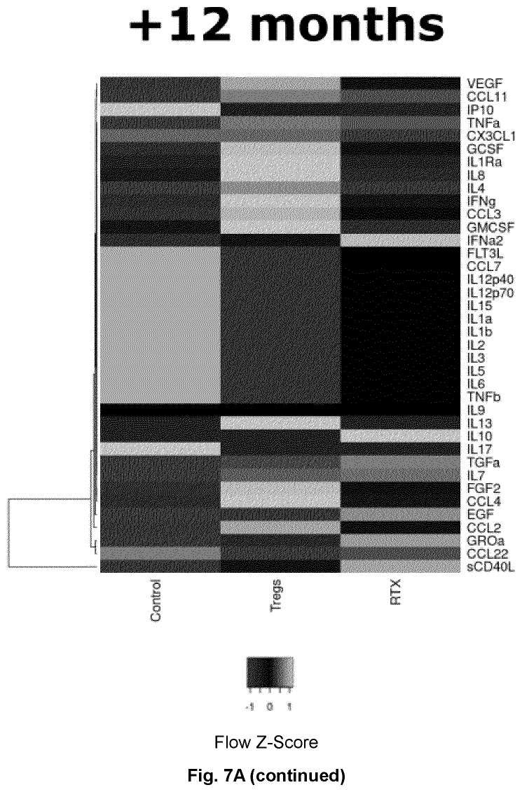

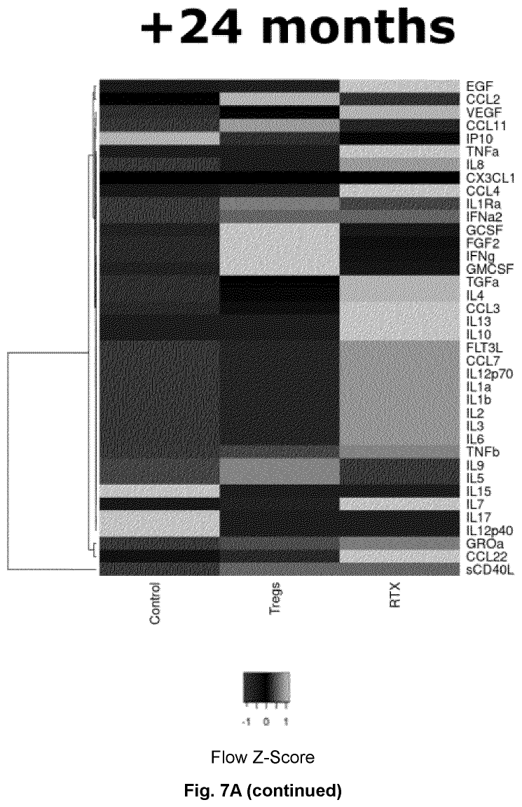

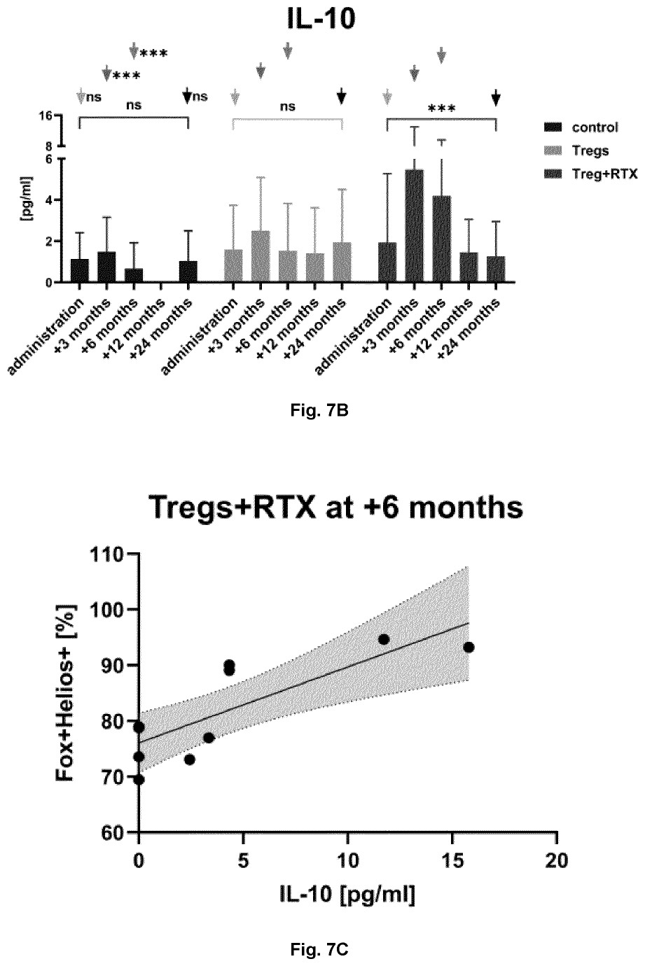

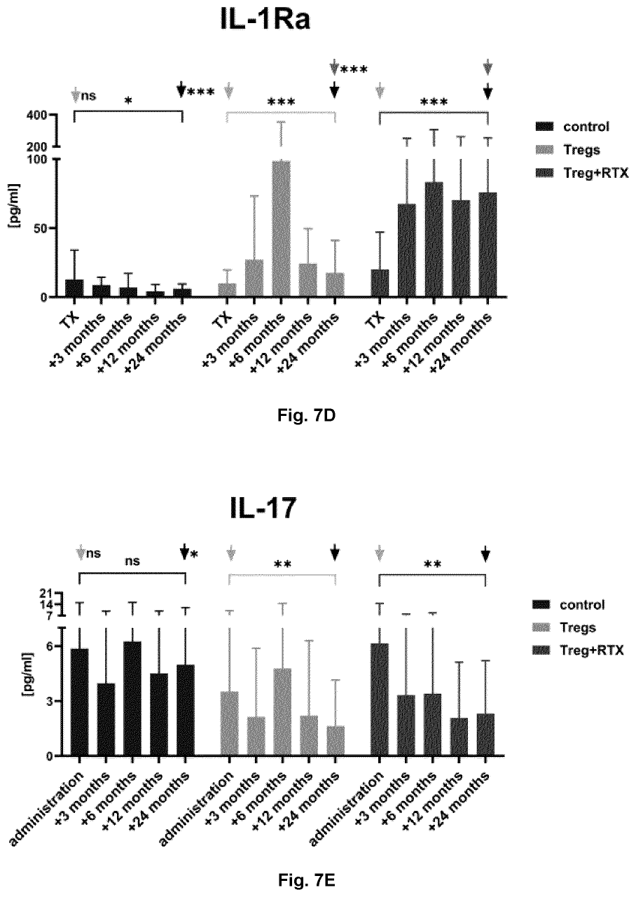

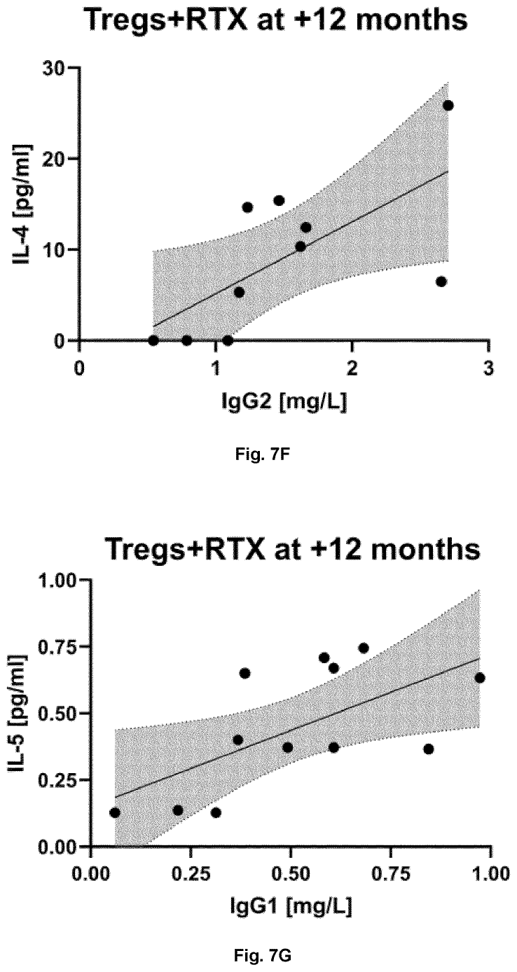

FIG. 7 Serum cytokines network during patient follow-up.

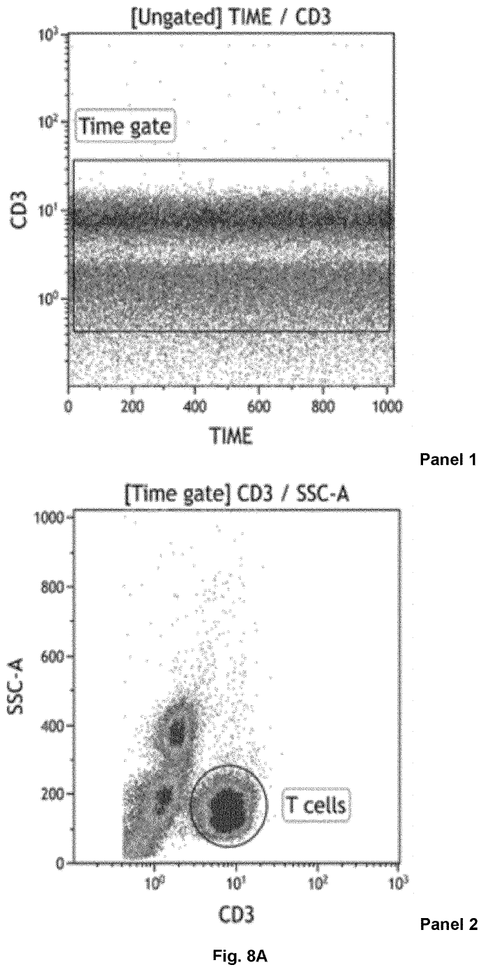

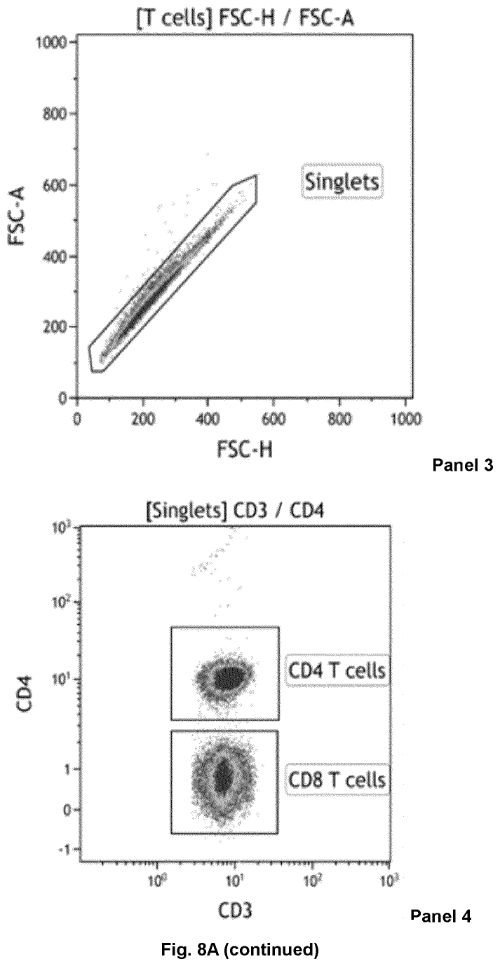

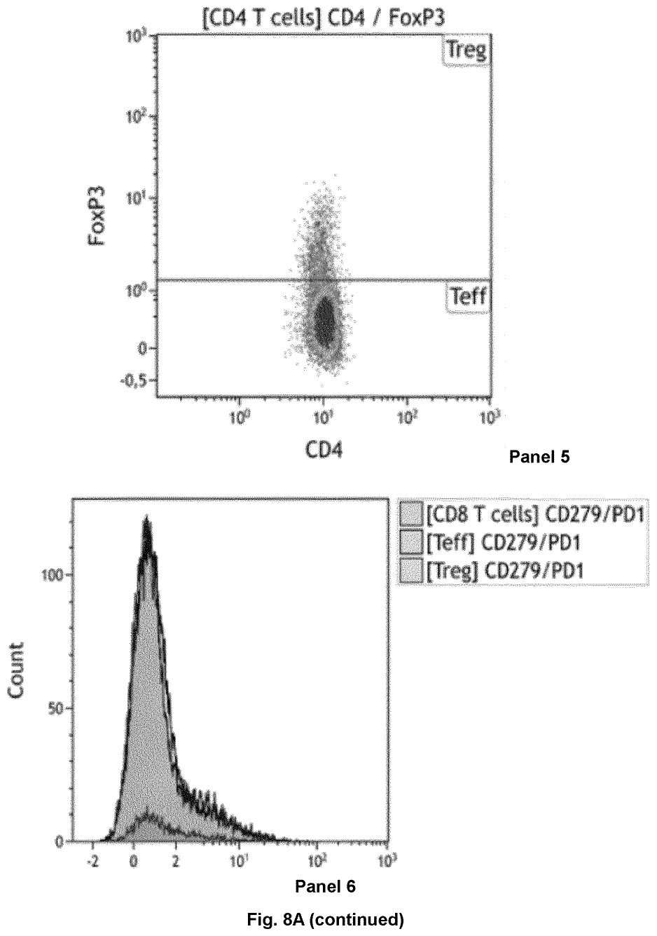

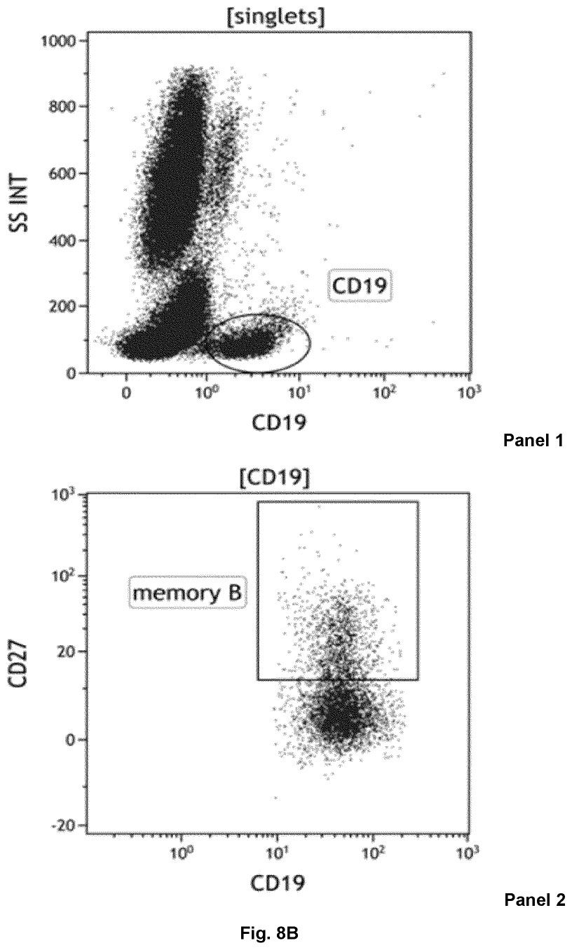

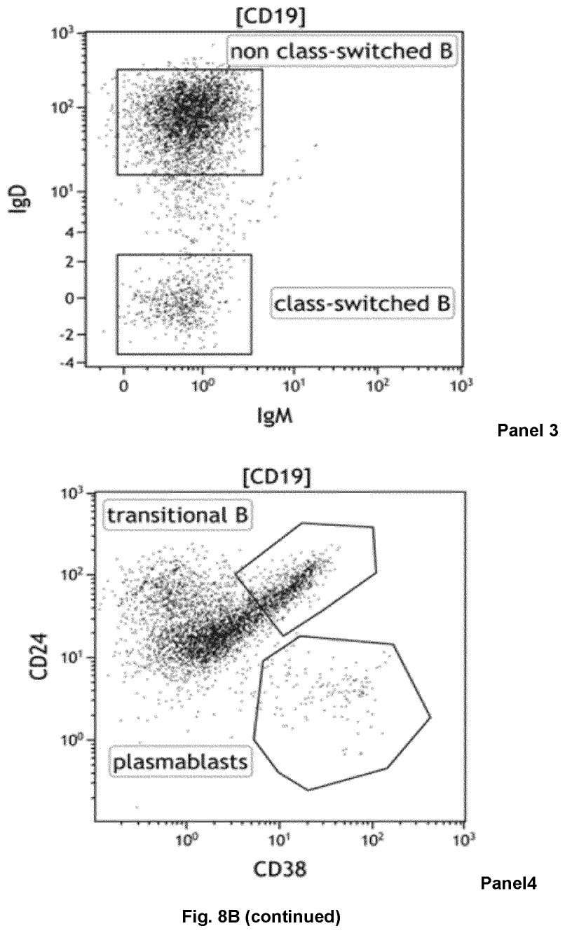

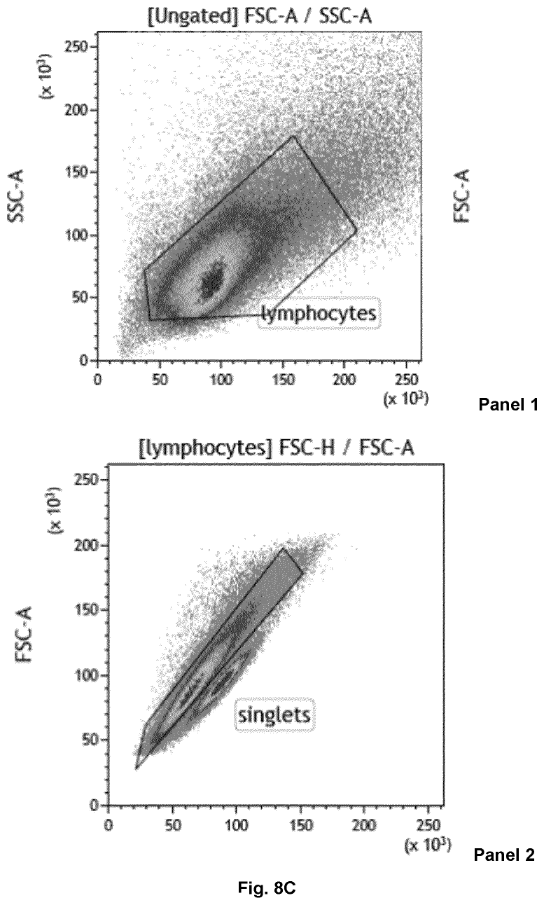

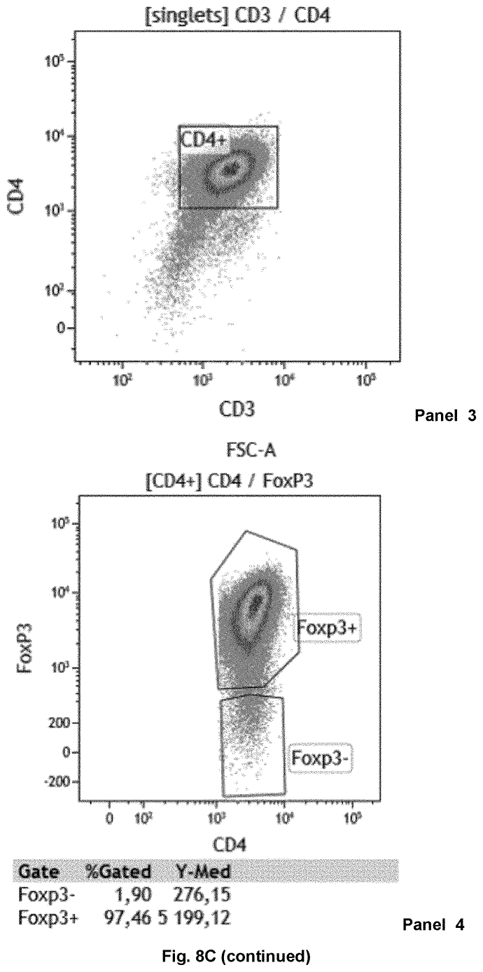

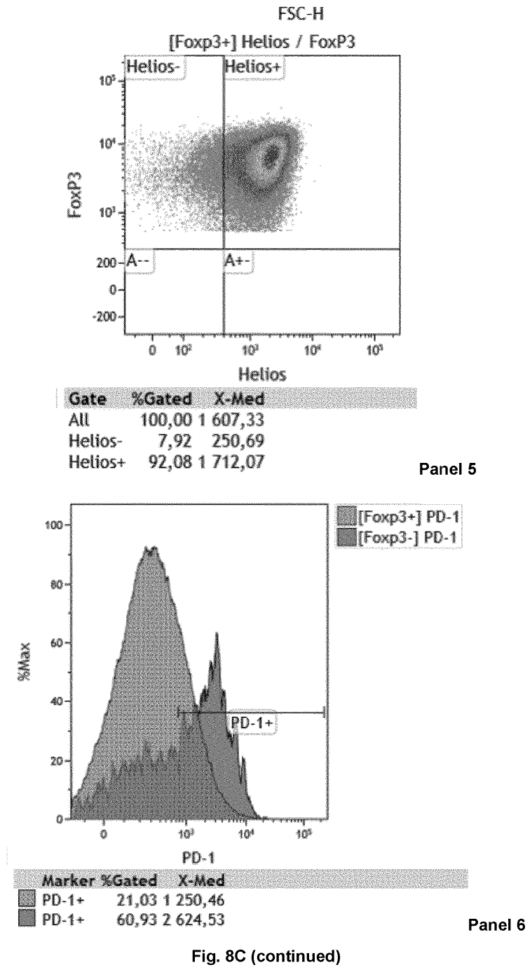

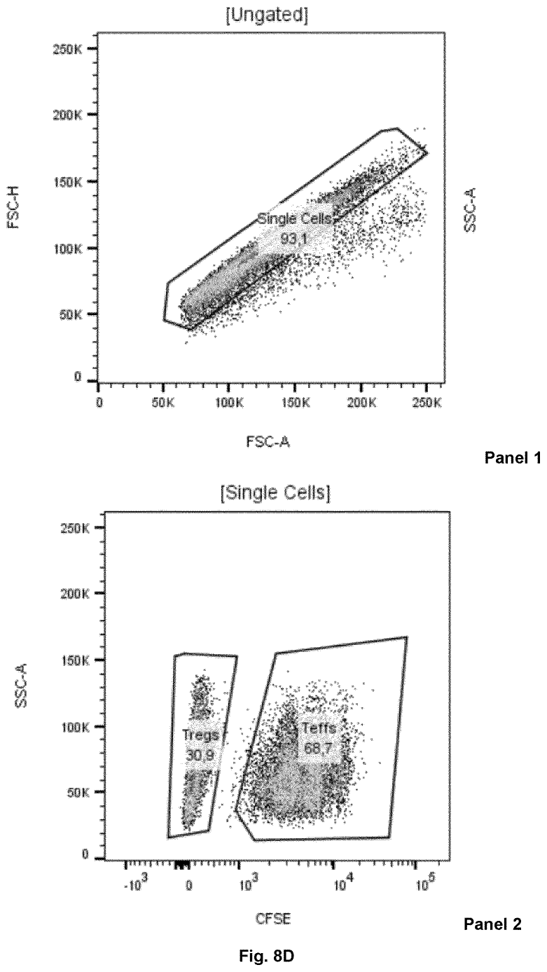

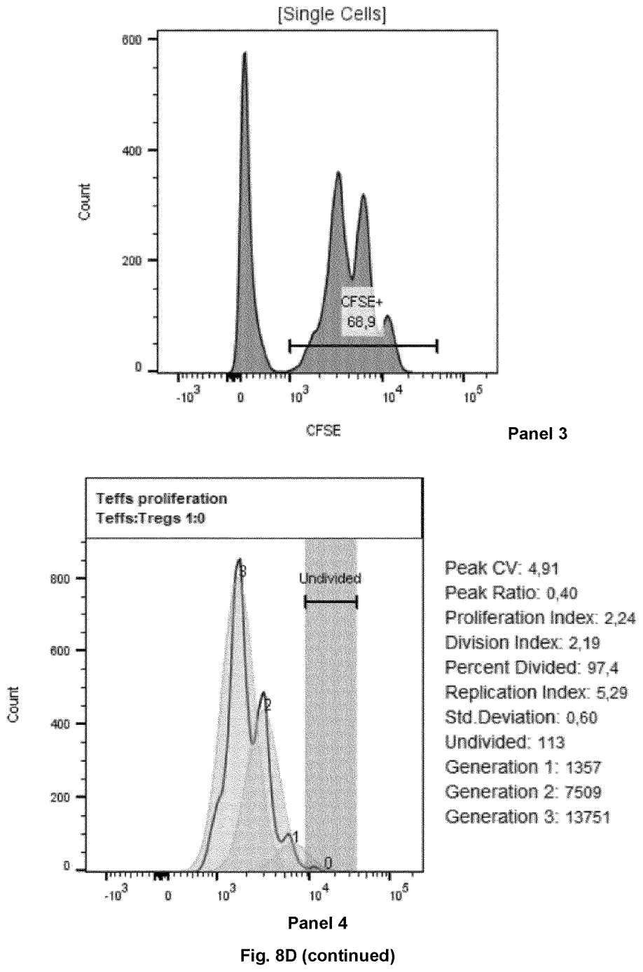

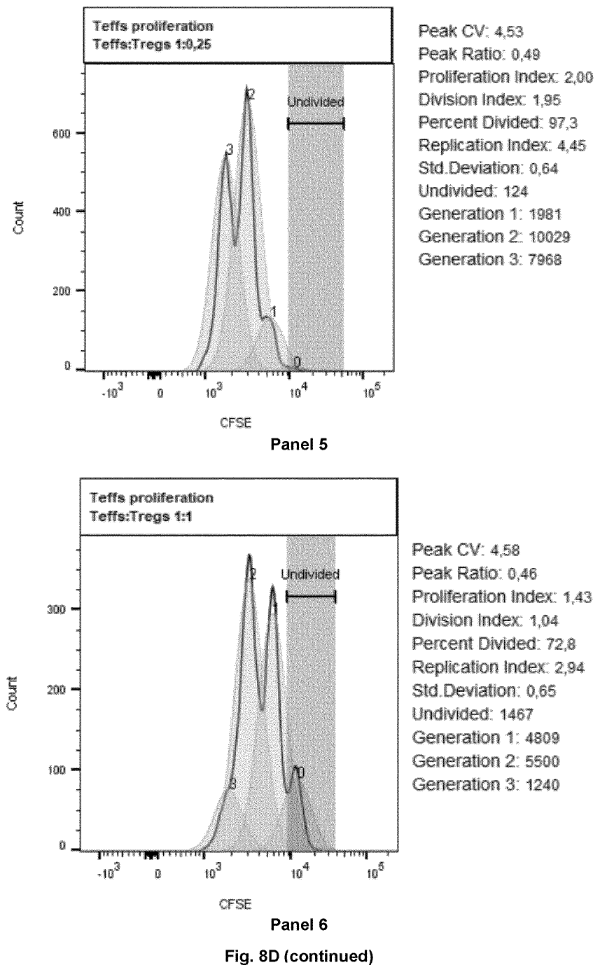

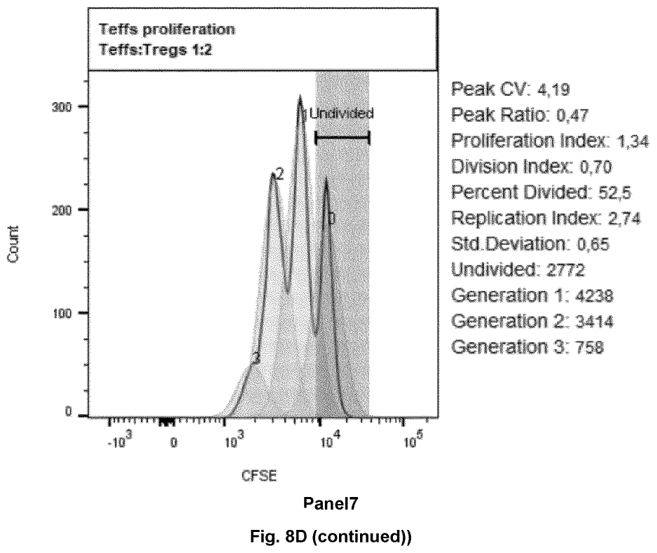

FIG. 8 Flow cytometry gating strategy for single cell's antigen expression. (A) Flow cytometry gating strategy for single cell's antigen expression. A representative example of a surface expression of CD279 (PD-1) antigen on regulatory T cells (Tregs FoxP3+), effector CD4+ T cells, and CD8+ T cells is shown. (B) Flow cytometry gating strategy for single cell's antigen expression. A representative example of lymphocyte B subsets. (C) Flow cytometry gating strategy for single cell's antigen expression. A representative example of a surface expression of CD279 (PD-1) antigen on regulatory T cells in in vitro culture is shown. (D) Flow cytometry FCS gating strategy in FlowJo Software and a representative example of the proliferation modelling are shown.

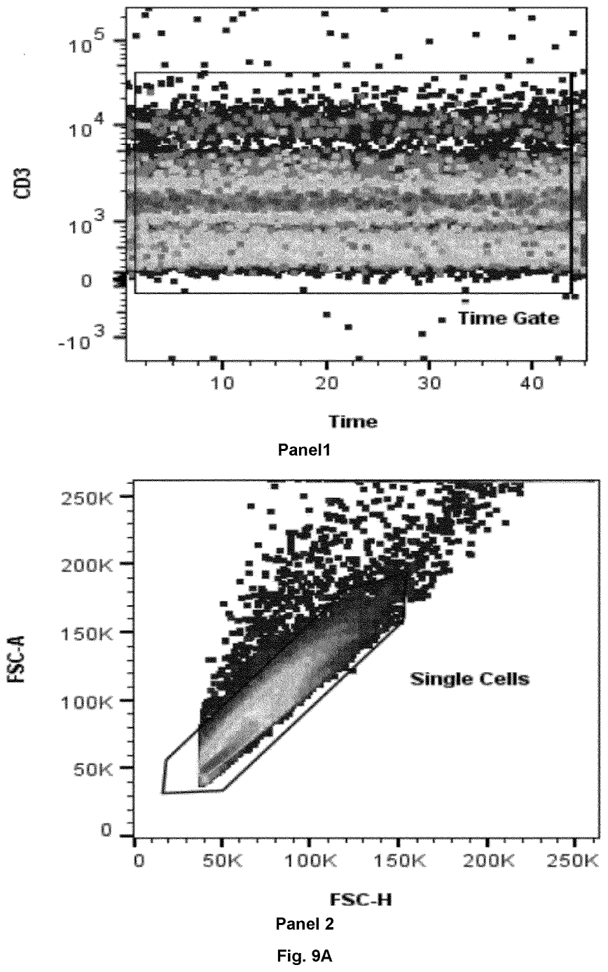

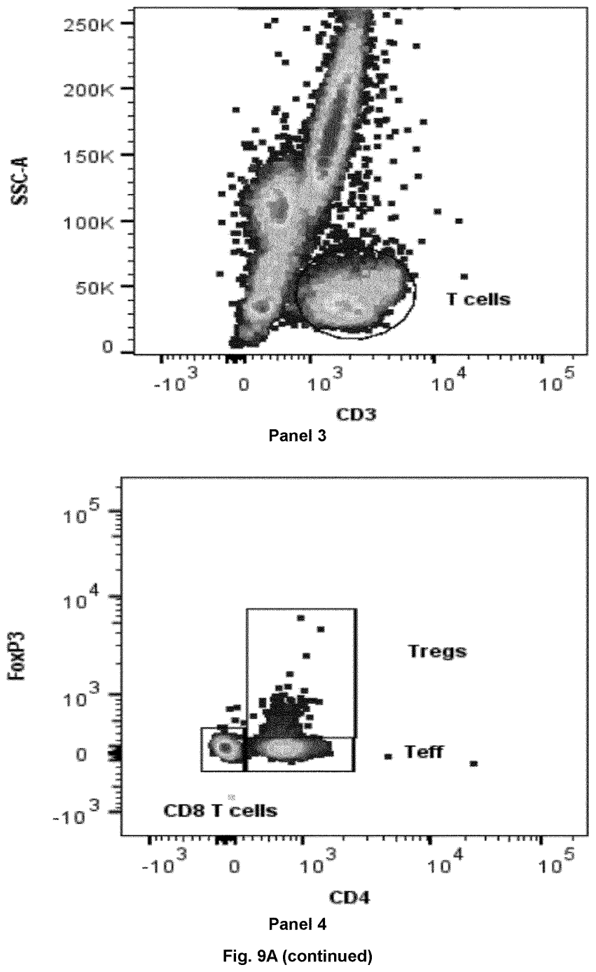

FIG. 9 Basic flow cytometry FCS gating strategy in FlowJo Software and scheme for the dimensionality reduction algorithms analysis. (A) Basic flow cytometry FCS gating strategy in FlowJo Software for the dimensionality reduction algorithms analysis. (B) Scheme for the dimensionality reduction algorithms analysis in the FlowJo Software.

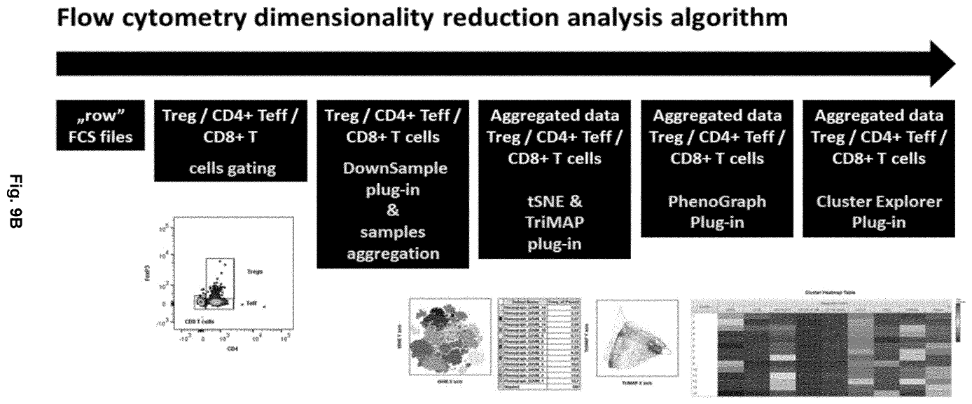

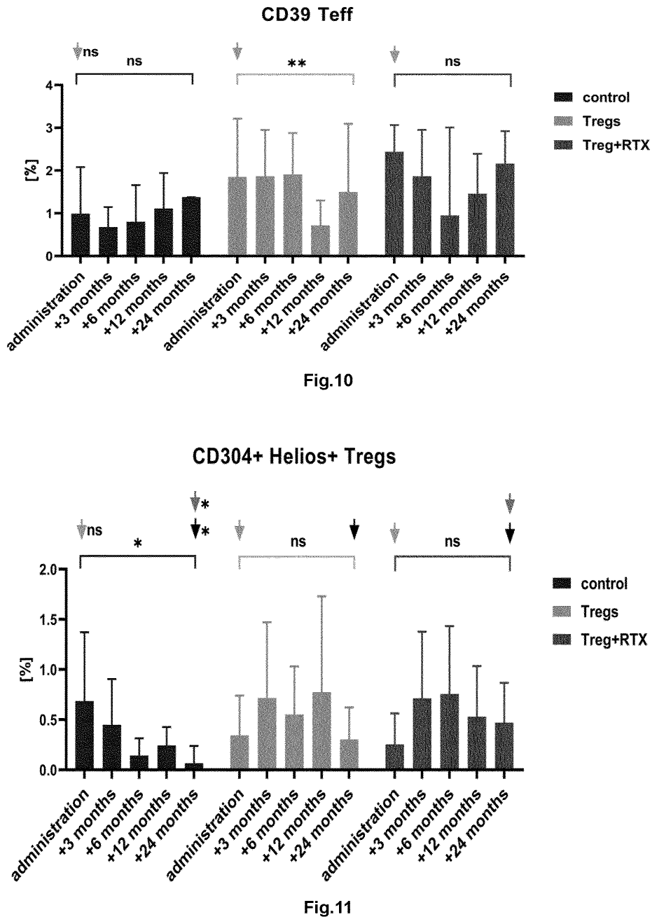

FIG. 10 Surface expression of CD39 on regulatory T cells (Tregs FoxP3+) and effector CD4+ T cells from administration up to 2 years follow-up.

FIG. 11 Surface expression of CD304 (NRP-1) on regulatory T cells (Tregs Helios+ FoxP3+) from administration up to 2 years follow-up.

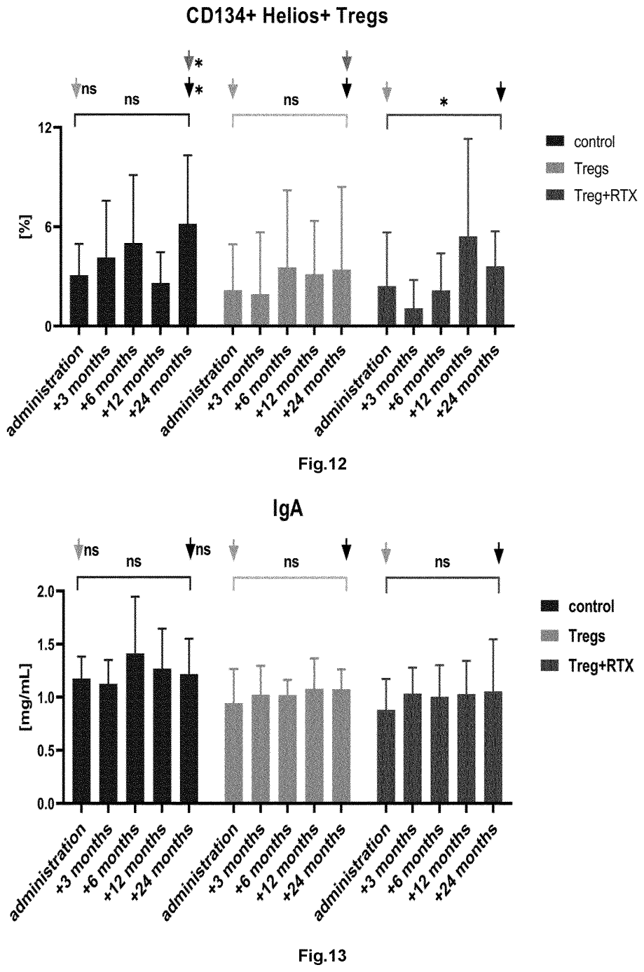

FIG. 12 Surface expression of CD134 (OX-40) on regulatory T cells (Tregs Helios+ FoxP3+) from administration up to 2 years follow-up.

FIG. 13 Serum IgA concentration from administration up to 2 years follow-up.

FIG. 14 Correlation of IL-1Ra serum concentration and a daily insulin dose per body weight (DDI/kg b.w.) at twelve months and glycated haemoglobin 24 months after administration.

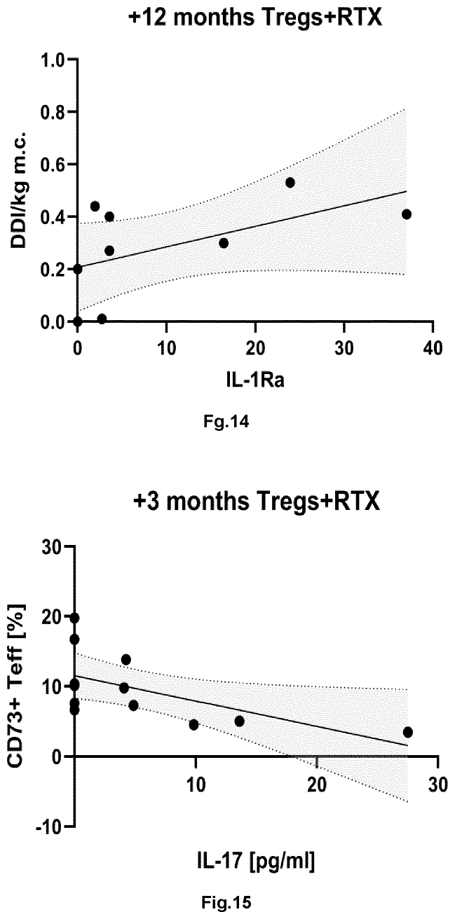

FIG. 15 Correlation of IL-17 serum concentration and surface expression of CD73 on effector CD4+ T cells at three months after administration.

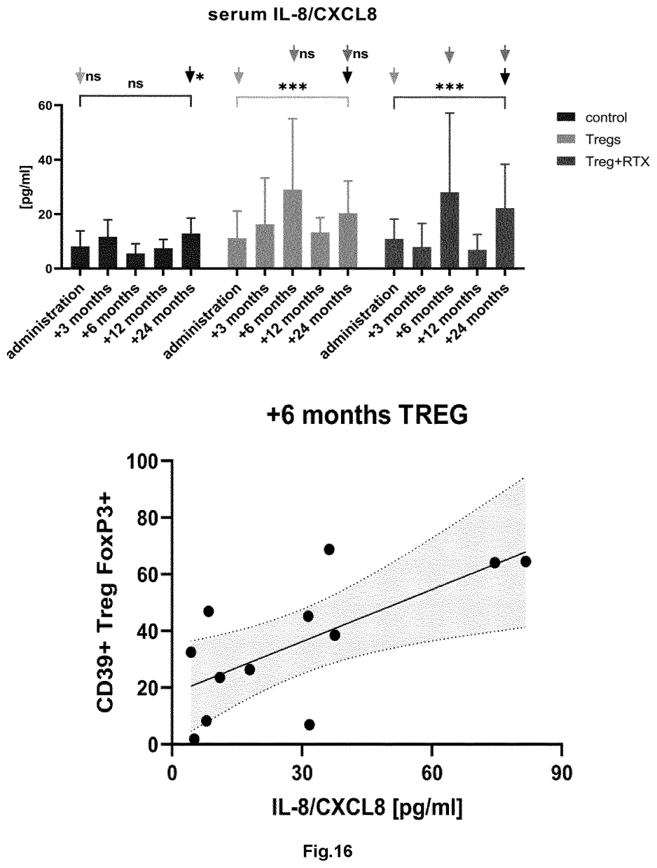

FIG. 16 Serum IL-8/CXCL8 concentration from administration up to 2 years follow-up, and correlation of IL-8/CXCL8 serum concentration and surface expression of CD39 on regulatory T cells (Tregs FoxP3+) six months after administration.

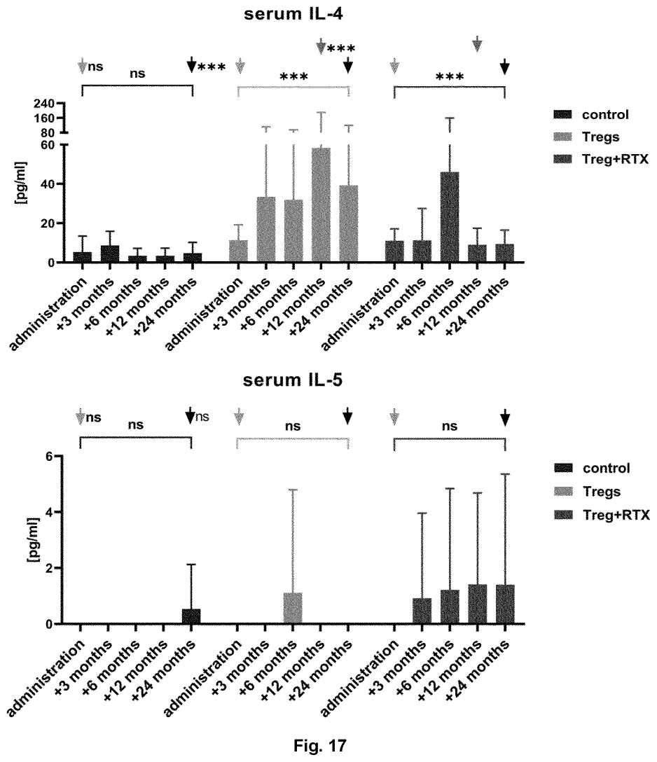

FIG. 17 Serum IL-4 and IL-5 concentration from administration up to 2 years follow-up.

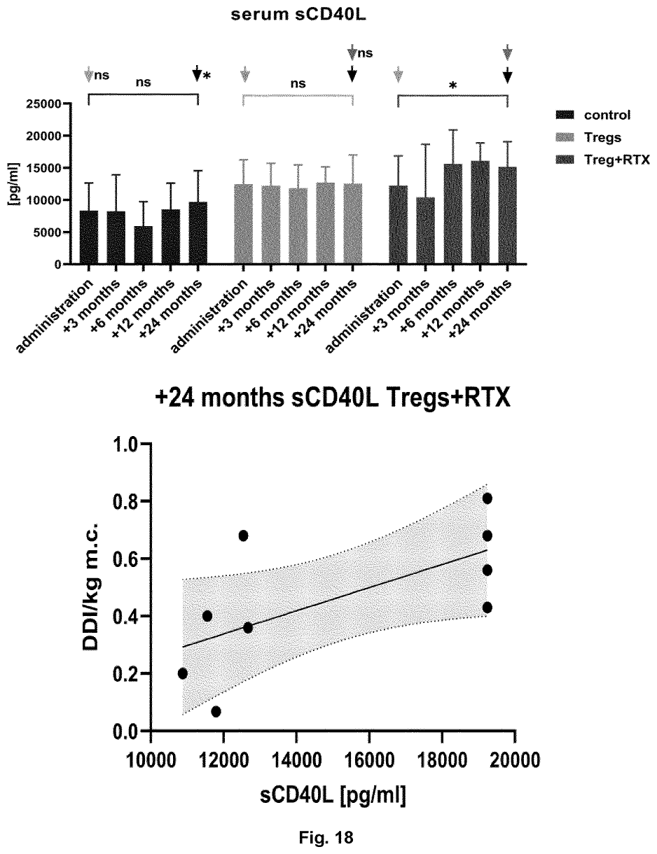

FIG. 18 Serum sCD40L concentration from administration up to 2 years follow-up, and correlation of sCD40L serum concentration and a daily dose of insulin per body weight (DDI per kg b.m.) at twenty-four months after administration.

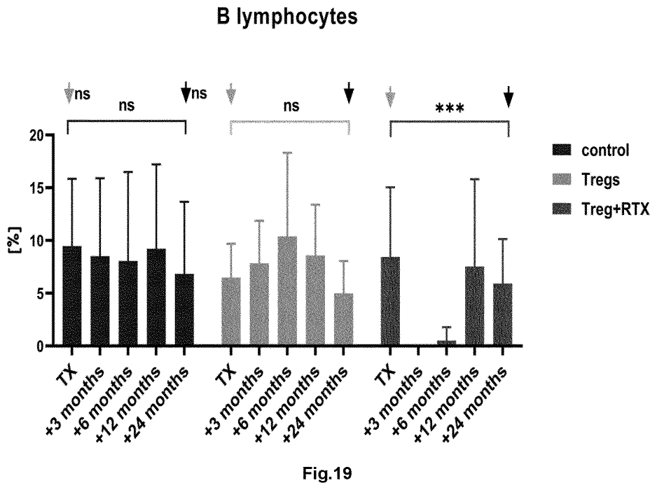

FIG. 19 Lymphocytes B from administration up to 2 years follow-up.

The present invention is further illustrated by the following non-limiting Example.

### EXAMPLE

### Introduction

This study according to the present Example aimed to identify and validate biomarker candidates for immune intervention in DM1 in the clinical trial TregVAC2.0 (clinical trial registration ISRCTN37116985). The inventors assessed cellular and humoral immunity in newly diagnosed DM1 patients treated with combined therapy of autologous polyclonal CD3+CD4+CD25highCD127− T regulatory cells and anti-CD20 antibody (Tregs+RTX group) as compared to the patients treated with polyclonal Treg administration only (Tregs group) or standard-of-care treatment with insulin (control group). As a final result, we expected to find a marker or markers in the immune system which correlate with β-cell function and can be used to predict the response to the immunotherapy used in the trial (FIG. 1).

### Materials and Methods

### Study Design

The present inventors have performed a phase 1/2, prospective, multicenter clinical trial to study efficacy and impact on selected immune parameters of combined therapy with autologous Treg administration and anti-CD20 monoclonal antibodies in children and adolescents with recently diagnosed DM1. The study was a prospective, open-label and randomised 24-months-lasting clinical trial registered as ISRCTN37116985 and EudraCT 2014-004319-35. In this three-arm trial, the follow-up was performed in a standard-of-care control group (control; 11 patients), the group treated with autologous polyclonal Treg only (Tregs; 12 patients), and the group treated with combined therapy with autologous polyclonal Treg and anti CD20 antibodies (Tregs+RTX; 13 patients). Interventional groups, which consisted of Tregs+RTX and Tregs patients, received two doses of Treg in an open-label manner, 30×10⁶ cells/kg b.w. per dose, three months apart starting from day 0. The administration of the anti-CD20 antibody was blinded and placebo-controlled. The patients were randomly assigned to an anti-CD20 antibody or placebo by the element of chance (coin) to receive four dosages of rituximab (Tregs+RTX group) or placebo (Tregs group) on +14, +22, +29, and +36 days of the trial. All the participants were followed-up for two years posttherapy and assessed at administration, three, six, twelve, and twenty-four months after, as given in the study flow diagram shown in Figure. The report on the efficacy and safety of combined therapy made in this clinical trial has already been published. We have proved that the combined autologous Treg administration and anti-CD20 antibodies therapy was superior to the Treg administration-only treatment in DM1, as assessed by the AUC of C-peptide mixed meal tolerance test and the percentage of patients in clinical remission at 24 months of the trial. The therapy was safe, despite the 80% adverse events rate in the Tregs+RTX patients, as no adverse event led to the withdrawal of the intervention or the patient's death (7).

The study according to the present invention aimed to assess the immune imprint in DM1 individuals. In a two-years-lasting follow-up, we set multicolour immunophenotypes of CD4+ Treg and CD4+ T Teff and CD8+ T cells, trying to correlate them with the clinical and laboratory outcomes. Likewise, immune correlations were analyzed in humoral immunity and cytokine milieu. The results were then matched with the in-vitro model of the Treg and CD4 Teff cell cultures from DM1 patients and healthy volunteers, as given in the study flow diagram (FIG. 1). Finally, the laboratory findings were correlated with the trial's clinical outcomes. The study was approved by an independent institutional review board (NKBBN/374/2012-NKBBN/374-7/2014 for the clinical trial and NKBBN/414/2018 for the in-vitro study) and all participants signed an informed consent form.

### Patients

All the participants were recruited based on the detailed inclusion and exclusion criteria for both the clinical trial and the in-vitro model (Table 1a, 1b and 2). By the power analysis for sample numbers, thirteen patients in the randomised treatment arm were satisfactory to find a 20% difference in the geometric mean ratio of AUC (0-240 min) of C-peptide (alpha 5%), a significant outcome of the clinical experiment. For the in-vitro model, 12 DM1 patients and 12 healthy controls were enrolled. DM1 patients were selected upon inclusion/exclusion criteria equal to the clinical trial, and healthy controls were blood donors from the Regional Blood Bank in Gdansk whose buffy coats left after blood product preparation. The detailed baseline demographics are given in Tables 1A and 1B for the clinical trial and the in-vitro model.

### Methods

### Cell Isolation

The Ficoll Paque Plus (GE Healthcare, Chicago, IL), a density gradient isolation, was used for PBMC isolation from EDTA whole blood from DM1 patients or buffy coats from blood products prepared by healthy volunteers. The isolated cells were then counted and tested for viability with the Bio-Rad TC20 (Hercules, CA), an automated trypan blue cell viability analyser. The minimum viability cut-off used for testing was 90%.

### Cell Sorting and Culture for In Vitro Model of Treg Stimulation

CD4+ T cells were isolated from PBMC using a negative immunomagnetic selection kit (EasySep Human CD4 Negative Selection Kit, StemCell Technologies; Vancouver, BC, Canada) and next stained with fluorescence-conjugated monoclonal antibodies (BDBiosciences, Poland): anti-CD3 (clone UCHT1), anti-CD4 (clone SK3), anti-CD25 (clone M-A251), and anti-CD127 (clone HIL-7R-M21). Finally, cells were sorted with FACS Aria II sorter or FACS Influx sorter (BD Bioscience, Franklin Lakes, NJ) into regulatory T cells (Treg), with the phenotype (CD4+/CD25high/CD127−/doublets−) and effector T cells (Teff) with the phenotype (CD4+/CD25low/CD127+/doublets−). The sorted cells were then cultured according to the previously published protocol (34, 35). Briefly, cells were suspended in X-Vivo medium (Lonza, Belgium) supplemented with a heat-inactivated autologous serum (DM1 patients) or AB human male serum (healthy controls) and 1×10⁴ UI/ml of IL-2 (Proleukin, NOVARTIS, Germany) for 12 days. Treg and Teff were activated for proliferation with beads coated with anti-CD3 and anti-CD28 antibodies (MACS GMP ExpAct Treg Kit, Miltenyi Biotec, Germany) at day 0 and day 5 in 1:1 bead-to-cell concentration ratio.

### Flow Cytometry

An extended flow cytometry profile of peripheral lymphocytes was applied to study several immune parameters for the clinical trial groups, as given in Table 3. Twenty-two markers have been analysed using three 16-colour combinations of the fluorochromes. We have used minimum backbone markers for gating purposes, which refers to the CD3, CD4, FoxP3, Helios, CD45RA, and CD62L markers. Upon this, Treg, CD4+ Teff, and CD8+ T cells were gated, as shown in FIG. 8A.

Peripheral blood lymphocytes B were gated as CD19/CD20 double positive lymphocytes (backbone markers) and further analysed for the antigens associated with the cell's maturation, memory development, and class-switching (FIG. 8B), as given in Table 3.

Appropriate isotype controls and fluorescence minus one (FMO) approach were used to gate the population of interest for every analysis. The minimum number of cells per flow cytometry tube was 200.000±20.000 viable cells, and a minimum of 80.000 out of it was collected upon flow cytometry testing with BD LSRFortessa Cell Analyzer (BD Bioscience, Franklin Lakes, NJ, USA).

### Suppression Assay

Eight DM1 and five healthy control cultures were randomly selected to perform the suppression assays on day seven and day twelve of culture. The suppressive potential of Tregs was assessed as inhibition of Teff proliferation in the presence of Treg. The 1×10⁴ Teff per culture were stained with 1 μM/ml of CFSE proliferation dye (BD Bioscience, Franklin Lakes, NJ) and cocultured with titrating concentrations of Treg as follows (Teff:Treg) 1:2, 1:1, 1:0.5, 1:0.25, and 1:0.125. Cells were then activated with magnetic beads coated with anti-CD3 and anti-CD28 antibodies (MACS GMP ExpAct Treg Kit, Miltenyi Biotec, Germany) in a 1:1 bead to Teff ratio and cultured for 72 h. The bead-stimulated cultures of Teff without Treg served as a positive control, and the unstimulated cultures of Teff and Treg as negative controls. The fluorescence of CFSE in the samples was acquired with BD LSR Fortessa Cell Analyzer (BD Bioscience, Franklin Lakes, NJ). Results were analysed using the proliferation modelling tool in FlowJo (Ashland, OR) and presented as proliferation index (PI) and a representative gating example is given in FIG. 8D.

### Flow Cytometry Data Analysis

Several immune parameters of Treg, CD4+ Teff, CD8+ T cells, and B lymphocytes were analysed. First, we screened data using the heatmap approach for each cohort up to two years of the follow-up (FIG. 2A). Next, with stochastic analysis of cell phenotype, a further assessment was done to find the best fitting parameters, allowing to discriminate treated from control patients. Finally, ANOVA statistics were calculated for the selected parameters to find statistical significance.

The flow cytometry data were analysed using Kaluza Software (Beckman Coulter, Brea, CA) and FlowJo Software (V10, Ashland, OR, USA). First, using backbone markers, significant T and B cell subsets were identified, and the expression of distinct antigens was noted as percentages. Next, data were analysed using FlowJo's Software dimensionality reduction algorithms to find significant cell subpopulations between tested cohorts. We used t-Distributed Stochastic Neighbor Embedding (tSNE) and TriMap algorithms with the following PhenoGraph algorithm, a clustering method for identifying phenotypically distinct subpopulations. (37, 38, 39)

In FlowJo software, starting with the row FCS files, downsampling was done to reduce the number of events to normalise the population size, and then Treg, CD4+ Teff and CD8+ T cells were gated (FIG. 9A). We used 30.000 events downsampling regularly across the time order of collected events. Next, normalised Treg, CD4+ Teff and CD8+ T cells subpopulation events from single samples were aggregated into one FCS file (particular condition) for further dimensionality reduction algorithms analysis. The tSNE was done using opt-SNE configuration, setting: iteration=1000, perplexity=30, the k nearest neighbours algorithm as a vantage point tree, and Barnes-Hut as a gradient algorithm (37). The TriMap analysis was done with Euclidean distance function, nearest neighbors=10, and the number of outliers=5. (38) Then, the PhenoGraph algorithm was calculated with the k number (the number of nearest neighbours used for the nearest-neighbour graph) suggested by the plugin upon FCS file structure. (39) Finally, the Cluster Explorer plugin was used to select populations of interest. The detailed analysis protocol is given in FIG. 9B.

### Serum Immunoglobulins and Autoantibodies

The serum concentration of IgA, IgM immunoglobulins, and IgG subclasses: IgG1, IgG2, IgG3, and IgG4 were measured with Bio-Plex Pro Human Isotyping Panel kit (Bio-Rad, Hercules, CA) according to the manufacturer's protocol and read on Luminex MAGPIX analyser (Merck Millipore; Burlington, MA, USA).

### Autoantibodies

Anti-GAD65 (glutamic acid decarboxylase antibodies) and anti-IAA (insulin autoantibodies) antibodies were tested with ELISA (Euroimmun, Germany). Anti-ICA (anti-islet cell antibodies) were tested with indirect immunofluorescence assay (IIF) with primate pancreas as the antigenic substrate (Euroimmun, Germany).

### Serum Cytokines

The serum concentration of 38 cytokines was measured with the bead-based multiplex assay—Human Cytokine/Chemokine Magnetic Bead Panel, Milliplex (Merck Millipore; Burlington, MA) according to the manufacturer's protocol and read on Luminex MAGPIX analyser (Merck Millipore; Burlington, MA).

### Statistical Data Analysis

Data were expressed as medians with a standard deviation. Only cleaned-up data with the Grubbs test were used for all statistical tests. All between-group comparisons were made using the nonparametric Mann-Whitney U test, while Kruskall-Wallace or Welch's ANOVA was applied for multiple data sets. If data were skewed, not Gaussian, the Brown-Forsythe ANOVA test was used. The relationships between data sets were tested with Spearman rank correlations, and the frequency was assessed with the Chi-square test. The visualisation of correlations calculated for several data sets was done with colour-coded correlation matrix graphs and XY data points diagrams with 95% confidence bands of the best-fit line. The Monte Carlo simulation did the Principal components analysis (PCA). A receiver operating characteristic curve (ROC) was calculated with Wilson/Brown method and 95% confidence interval (95% CI). Data were presented as medians with an interquartile range and visualised with bar graphs or individual values. The tops of each bar indicate means, while lines represent standard deviation. Significance was set at p<0.05. The significance code for the p-value was as follows: “***” (0,000-0,001), “**” (0,001-0,010), and “*” (0,010-0,050). All analyses were performed in Prism 9 (GraphPad Software; Boston, MA). The heatmaps were generated with a Heatmapper (http://www.heatmapper.ca/), where average linkage was used as a clustering method and Euclidean as a distance measurement method. (40)

### Results

### Treg Number, FoxP3+ and Helios Transcription Factors Expression

No significant differences were found between the groups throughout the follow-up (FIG. 2A). Neither FoxP3+ or FoxP3+Helios+ Treg percentage changed significantly in control or treated patients. No between-group differences were noted throughout the 2-year-lasting monitoring (FIG. 2B, 2C). With a principal component analysis of FoxP3+ or FoxP3+Helios+ double positive Treg, only 11-20% of differences between treated and control groups were tested from recruitment up to two years of monitoring (FIG. 2D).

On the contrary, compared to a stable percentage in healthy controls (HC), we have found a decreasing percentage of FoxP3+ Treg and FoxP3+Helios+ Treg between day seven and day twelve in the cultures of cells from DM1 individuals in our in-vitro model of Treg stimulation (FIG. 2E, G). Similarly, the expression (measured as MFI) of FoxP3 decreased throughout the culture, while Helios expression declined in the DM1 group only (FIG. 2F, H).

### PD-1, the Immune Checkpoint Antigen

An in-depth analysis revealed that the percentage of PD-1+ T cells changed significantly throughout the study (FIG. 3A-C). In the control group, the percentage of PD-1+ T effector cells (Teff) gradually decreased from +6 to +24 months of the follow-up (p=0.02). Conversely, there was an increasing percentage of PD-1+ CD4+ Teff and PD-1+CD8+ T cells in the treated groups throughout the trial; however, it was significant only in the combined treatment Tregs+RTX group (FIG. 3B, 3C). Hence, there was a considerable difference between the control and combined-treatment groups in the percentage of PD-1+ Teff at +24 months (p=0,009) (FIG. 3B, black arrows) and PD-1+CD8+ T cells at +12 months (p=0.04) (FIG. 3C, brown arrows). There were similar trends in the follow-up in the level of PD-1+ Treg, but neither the decrease in the control group nor the increase in the treated group reached statistical significance. The only between-group difference for Treg was between control and combined-treatment patients at +24 months (p=0,001) (FIG. 3A, black arrows). In-vitro, compared to healthy controls, a higher percentage of PD-1+ Treg and PD-1+ Teff was noted in the DM1 group at day 0 (p=0,032 and p=0,006), and PD-1+ Treg subsequently increased in the culture in both groups to be comparable at day +7 (p=0,932) (FIG. 3D, F). Interestingly, on day +12, the percentage of PD-1+ Treg in cultures dropped dramatically in the cultures from DM1 patients and was significantly lower than in the healthy group (FIG. 3D). At the same time, the expression of PD-1 on a per-cell basis measured as MFI was markedly higher in the cultures from DM1 patients than in the ones from healthy controls at day 0, day +7 and day +12 (FIG. 3E). No such differences were noted in the cultures of CD4+ Teff (FIG. 3G).

Treg Suppressive Activity Correlated with the Percentage of PD-1+ Treg in the DM1 Group.

The suppressive activity of Treg was tested as an inhibition of the proliferation of Teff in the cocultures of stimulated Teff with autologous Treg. Interestingly, there was a correlation between the percentage of PD-1+ Treg and suppression only for the DM1 group at day +7 (r=−0,552; p=0,005) (FIG. 3H). The effect disappeared on day 12 when the percentage of PD-1+ Treg dropped significantly in the DM1 group (FIG. 3D, I). No correlation was observed for healthy controls, neither on day +7 or day +12 (FIG. 3H, I).

### The Expression of Other Biomarker Candidates in the Follow-Up

For Treg and Teff, a graduate loss of the percentage of CD73+ cells was found in control DM1 individuals (p=0,003 and p=0,007, respectively). The patients receiving Tregs or combined treatment did not manifest such a decrease (p>0.05) (FIG. 4). No differences were found in the percentage of CD39+ Treg or Teff (another enzyme involved in nucleotide metabolism) throughout the trial (FIG. 10). Similarly to CD73+ Treg, the percentage of CD304+(NRP-1 antigen) Helios+ Treg decreased in the control group only in the follow-up (p=0.02). Nevertheless, the expression of this biomarker on Treg was low, rarely exceeding 2% of all Treg (FIG. 11). Finally, a significant increase in CD134+(OX-40 antigen) Helios+ Treg percentage was noted in the control group and both interventional arms throughout the follow-up, reaching the highest level in the control group (FIG. 12).

### B Cell Subsets and Immunoglobulins

Significant changes were noted in the Tregs+RTX group only (FIG. 5A). Almost complete depletion of B cells was noticeable in the first six months after injection of the anti-CD20 antibody. At +12 months, as compared to the baseline, B cell recovery was seen with a significant reduction in the percentage of CD27+ B memory cells (p<0,001) (FIG. 5B). At the same time, the percentage of Breg-like cells (CD38++CD24++) and transitional (CD38+CD24+) B cells almost doubled (p<0,001). At +24 months, it dropped to baseline in the case of the former subset, but it was kept folded for the latter one (FIG. 5C, D).

In addition, a characteristic switch in the IgG1/IG2 index was found in the Tregs+RTX group. A significant decrease in the serum concentration of IgG1 (p=0,003) was noted, with a concomitant increase in the concentration of IgG2 throughout the follow-up (FIG. 5E, F). The levels of IgM were significantly reduced for the entire study up to +24 months (p=0,001), and the lowest levels were noted at +6 months of the trial (FIG. 5G). There were no changes in the serum concentrations of IgA (FIG. 13). No incidents of late hypogammaglobulinemia were observed one to two years after rituximab treatment for IgA (<0.42 mg/ml) and IgG (IgG1<3.16 mg/ml; IgG2<0.86 mg/ml; IgG3<0.14 mg/ml; IgG4<0.01 mg/ml) subclasses. (12) There was also a partial reduction in the serum autoantibodies, like anti-GAD-65 autoantibodies, that decreased significantly in Tregs+RTX patients only (p<0,001). On the contrary, anti-IAA autoantibodies were reduced in all patients at month +24, while anti-ICA autoantibodies did not change throughout the 24-month-lasting monitoring (FIG. 5H, I, J).

### The Correlation of Immune Markers and Disease Progression

The presented immune parameters were then correlated with the clinical outcome results, such as mixed-meal tolerance test (MMTT) assessed by area-under-curve of plasma C-peptide concentration (AUC), glycated haemoglobin (HbA1C), serum c-peptide, and daily dose insulin per kg of body weight (DDI/kg of b. w.). There was a common correlation of better clinical outcomes with the percentage of PD-1+ Treg and PD-1+ Teff in both control and treated patients throughout two years of the follow-up (FIG. 6).

Less commonly, the clinical parameters were correlated with the percentages of CD73+ Treg and CD73+ Teff throughout two years of the follow-up. Interestingly, there was also a correlation between CD304+ Treg and better clinical outcomes (FIG. 6).

The area under the curve (AUC) for a receiver operating characteristic curve (ROC) was calculated, and only for Tregs+RTX patients the significant markers were identified as PD-1+ Treg, PD-1+ Teff, and CD73+ Treg (Table 4). For the cut-off value of >16% of PD-1+ Treg and >8% of PD-1+ Teff, the sensitivity was 73% (43.44% to 90.25%; 95% CI), and 72% (45.25% to 89.50%; 95% CI) while specificity was 92% (64.61% to 99.57%; 95% CI), 90% (70.54% to 96.28%; 95% CI) respectively (FIG. 3A-C). For the percentage of CD73+ Treg for the >7% cut-off value, the sensitivity was 82% (52.30% to 96.77%; 95% CI), and specificity was 83% (55.20% to 97.04%; 95% CI), (FIG. 4).

### The Serum Cytokine Milieu

Serum cytokines were screened with the heatmap approach with no constant pattern in the groups throughout 2 years of the follow-up (FIG. 7A). Of note, compared to the control patients, IL-10 serum concentration was two-fold and three-fold higher in Tregs and Tregs+RTX patients, respectively at three months of the follow-up (p<0,001) (FIG. 7B, dark grey arrows). The phenomenon persisted up to 6 months post-recruitment (FIG. 6B, brown arrows). Moreover, IL-10 concentration was positively correlated with the percentage of FoxP3+Helios+ double positive Treg at six months post-recruitment in Tregs+RTX group (r=0,772; p=0,013) (FIG. 7C). The IL-1 receptor antagonist (IL-1Ra) was another anti-inflammatory cytokine upregulated in the sera of Tregs (p<0,001) and Tregs+RTX (p<0,001) patients, as compared to control ones (p=0,040) throughout the follow-up (FIG. 7D). Notably, six months after recruitment, IL-1Ra serum concentration was fourteen-fold higher in Tregs patients and twelve-fold higher in Tregs+RTX patients than in the control group (p<0,001). The phenomenon persisted until the end of the follow-up in Tregs+RTX patients, while it almost disappeared in the Tregs group at 24 months of the follow-up (FIG. 6D, black arrows). We then found a positive, weak correlation between the concentration of IL-1Ra and DDI/kg b.w. (r=0,671; p=0,028) at +12 months and glycated hemoglobin (r=0,834; p=0,009) at +24 months in Tregs+RTX group (FIG. 14).

In the case of proinflammatory cytokines, we have found a significant reduction in the concentrations of IL-17 throughout the study. While the levels of IL-17 were stable in the control samples throughout 2y follow-up (p=0,970), they gradually decreased in Tregs (p=0,003) and Tregs+RTX (p=0,006) groups (FIG. 7E). In addition, there was a negative correlation between CD73+CD4+ Teff and the concentration of IL-17 (r=−0,694, p=0,016), noted at +3 months in Tregs+RTX patients (FIG. 15).

Another upregulated cytokine in Tregs and Tregs+RTX was the chemotactic factor IL-8/CXCL8. In control samples, IL-8/CXCL8 was comparable throughout 2y follow-up (p=0,590), but in Tregs (p=0,040) and Tregs+RTX (p=0,010), an increased serum concentration was noted (FIG. 16). A positive correlation between CD39+ FoxP3+ Treg and IL-8/CXCL8 serum concentration (r=0,615, p=0,038) was indicated at +6 months in the Tregs group (FIG. 16).

The B cells class switching process was accompanied by the increased serum concentration of IL-4, IL-5 and soluble CD40 ligand (sCD40L) in Tregs and Tregs+RTX groups. The IL-4 was highly upregulated in the Tregs group from month +3 to 2y of the follow-up. In the case of IL-5, a peak in serum concentration was noted at +6 months in Tregs+RTX patients (p<0,001) (FIG. 17). IL-4 positively correlated with serum IgG2 (r=0,767, p=0,014) in the Tregs+RTX group at +12 months of the follow-up. Similarly, IL-5 was positively associated with serum IgG1 (r=0,753, p=0,019) in Tregs+RTX at +12 months post-recruitment (FIG. 7F, G).

Unlike in control or Tregs groups, the serum concentration of sCD40L was upregulated in Tregs+RTX (p=0,040), with the highest concentration at +12 months (FIG. 18). Moreover, sCD40L was positively correlated with DDI/kg b.w. (r=0,812, p=0,021) at the end of the trial (FIG. 18).

### DISCUSSION

The study according to the present Example attempted to identify the most accurate immune biomarkers of the efficacy of the therapy with Treg in DM1. In the TregVAC2.0 trial (clinical trial ISRCTN37116985), we followed immune parameters of cellular and humoral immunity and the cytokine network of newly diagnosed DM1 patients treated with combined therapy of autologous polyclonal Treg and anti-CD20 antibody. The data were compared to monotherapy with polyclonal Treg and control standard-of-care patients treated with insulin only. We have found that the increased percentage of PD-1+ cells in CD4+ Treg, CD4+ Teff and CD8+ T cells from the peripheral blood was associated with favorable outcomes of the therapy. In vitro, the higher percentage of PD-1+ Treg correlated with better suppressive activity in functional suppression tests after stimulation for seven days. The effect disappeared on day 12, with a significantly reduced percentage of PD-1+CD4+Treg in the culture. These correlations were not noted in the cultures of healthy controls. In addition, the B cell compartment was rebuilt toward a higher percentage of regulatory-like B cells at the expense of reduced memory B cells and the increased serum IgG2 at the cost of reduced serum concentration IgG1 in the group treated with combined therapy. These changes were accompanied by the reduced proinflammatory potential in the subjects receiving treatment compared to control patients, measured as increased concentrations of serum IL-10 and IL-1Ra and decreased concentrations of IL-17.

A very important finding of the study according to the present Example was that the administration of polyclonal Treg preserved the expression of PD-1, and the combined therapy improved it even more, which may be a substantial therapeutic effect of this treatment. The percentages of PD-1 in T cells were higher in CD4+ Treg, CD4+ Teff and CD8+ T cells in DM1-treated patients compared to the DM1 control group at the end of the trial. Compared to the baseline, the expression of PD-1 was reduced in the DM1 control group, while not affected in Tregs group and increased in Tregs+RTX group. It was possible to calculate a PD-1+ cut-off value for both regulatory and effector T cells to monitor therapy outcomes. The cut-off above 16% for PD-1+ Treg and >8% for PD-1+ Teff pointed to the remission state in the Tregs+RTX group. Similarly, the cut off above 7% for CD73+ Tregs also was associated with better outcomes of the therapy. These calculations prove that Treg and Teff phenotypes can be applied for individual therapy response monitoring. We then validated this observation in the in vitro model, comparing people with DM1 and healthy controls. This part of the study highlighted the highest importance of the expression of PD-1 on Treg in DM1. We have found that the expression of this receptor on Treg and the percentage of PD-1+ Treg increased significantly on day +7, and the percentage then dropped considerably on day +12 in the cultures from DM1 patients. (FIG. 3D-G). Interestingly, in the cultures from DM1 patients, the suppressive potential of Treg in the suppression assay mainly correlated with the percentage of PD-1+ Treg as it was boosted on day +7 and significantly diminished on day +12. No significant differences in the stimulated expression of PD-1 receptors were found in the cultures of CD4+ Teff, and no associations were seen in the cultures of healthy controls. It may suggest that the expression of PD-1 receptor on T cells guards against autoimmunity in DM1. The disease is a stimulus which upregulates the expression of PD-1 similarly to the stimulation in vitro. Unfortunately, Treg from DM1 patients can upregulate PD-1 only transiently; therefore, the suppressive effect disappears quickly, and the disease may progress (FIG. 3H-I). It is an exciting observation in DM1 as the expression of PD-1 antigen on activated T and B cells is known to control the function and proliferation of T cells. (13, 14) The present study proves the link between this mechanism and protection from autoimmunity, such as DM1. There are reports that around one-third of cancer patients treated with PD-1/PD-L1 blocking antibodies developed immune-related adverse effects similar to autoimmune-like syndromes, for instance, autoimmune insulin-dependent diabetes. (15, 16) Also, in non-obese diabetic mice, a model of DM1, the blockade of PD-1 pathway resulted in rapid diabetes progression. (17) Furthermore, it was shown that the PD-1/PD-L1 axis controls only the early phase of diabetogenic effector T cells in the pancreas, giving indirect proof that only early intervention could slow down disease progression. (18) A similar observation was noted in systemic lupus erythematosus (SLE) patients treated with rituximab, where the percentage of PD-1^{high}CD4+ T cells decreased in the follow-up, which was associated with disease progression. (19) Finally, the expression of PD-1 in our study was the most commonly correlated with the clinical markers of β-cell function, and we have found several correlations for both treated and control patients. Moreover, the PD-1 associations were widespread for cell types (Treg, CD4+ Teff, and CD8+ T cells) and time points at follow-up (FIG. 6). The PD-1 upregulation was favorable for β-cell function, and as far as proved by the in-vitro model, its high expression correlated with the better suppressive function of Tregs.

Another important finding of the present Example was that PD-1 expression was the highest in the Tregs+RTX cohort, which might be attributed to anti-CD20 treatment. In autoimmunity, like idiopathic thrombocytopenic purpura (ITP), rituximab was shown to increase the number of Treg and the expression of Fas ligands, mRNA levels of the proteins involved in apoptosis BAX and BCL2, restored Th1/Th2 ratio, and TCR Vβ clonality. (29, 21) From the functional point of view, it was postulated that the depletion of CD20+ B cells changes T cells activation in several pathways, among which decreased antigen presentation could be critical. (22, 23) Alike in rheumatoid arthritis (RA), CD20+ B cells depletion reduced antigen-presenting cells (APC) pool and delayed autoimmunity but only to a certain degree. Once B cells returned, antibody production and T cell activation were restored, and the disease relapsed. (24, 25) B cell depletion inhibited antigen-specific CD4+ T cell expansion in the mouse models of arthritis and autoimmune diabetes, giving another example that B cells were essential for T cell responses. (26) We have noted in the Tregs+RTX group that B cell depletion resulted in an increased proportion of naïve, transitional, and regulatory-like B cells. Moreover, when the disease-specific autoantibodies were measured, the levels of anti-GAD65 persisted in control and Tregs groups but were significantly reduced in the Tregs+RTX group. (FIG. 5H).

Furthermore, the Tregs+RTX group was characterized by increased levels of anti-inflammatory cytokines, especially IL-10 and IL-1Ra, and reduced serum concentration of IL-17. It was also true for the Tregs group, but only for persistent IL-17 reduction, while a time-dependent increase in IL-1Ra and IL-10 was not upheld (FIG. 7B-D). It is concordant with other reports in which rebuilding of B cell compartment with rituximab resulted in fewer autoreactive clones and more B cell subsets capable of IL-10 production, mainly transitional B cells. (22)

In this light, the therapeutic strategy of the present Example to combine rituximab with the administration of regulatory T cells is well-founded, as B cell depletion reduced antigen presentation, induced pro-tolerant B cell phenotype and anti-inflammatory cytokine milieu. At the same time, Treg reduced T cell proliferation and promoted anti-inflammatory response. The expression of PD-1 can be adopted as a biomarker of this immunomodulatory therapy as its upregulation predicts the effect of the treatment (FIG. 3A, B).

In a previous clinical trial (TN-05) it was shown that four doses of rituximab preserved beta-cell function over one year, but when prolonged up to 30 months, no significant improvements could be found. (27, 29). Surprisingly, however, the present Example shows that a combination of rituximab with polyclonal Tregs resulted in the controlling of DM1 in terms of the better results of MMTT and fasting C-peptide levels, DDI/kg b.w., HbA1c, remission and insulin independence at two years follow up. The timing of B cell repopulation between six to twelve months after depletion (FIG. 19) and persistently decreased serum IgM levels were similar between TN-05 and TregVAC2.0 (FIG. 5G). Previously, there have been reported conflicting data on the effect of B cell depletion on the level of serum immunoglobulins: in the TN-05 trial, when compared to the control patients, DM1 patients treated with rituximab were characterized by comparable or increased serum total IgG concentration in the longer follow-up (27, 28), whereas some reports showed unchanged post-rituximab serum IgG1, IgG2, IgG3, IgG4 concentrations (29) or a selective decrease in serum IgG4 subclass only (30). In the study according to the present Example, however, once B cell repopulated in Tregs+RTX patients, an increase in IgG2 at the expense of IgG1 occurred (FIG. 5E, F). which is concordant with the concentrations of the class-switching cytokines (31) as IgG1 serum concentration was positively correlated with peripheral IL-4 and IgG2 with IL-5 around 12 months post-rituximab (the time of B cell repopulation). The switch could directly affect IgG function as IgG1 binds complement and FcR receptors on monocytes and neutrophils more than IgG2. (32) Hence, higher levels of IgG2 together with the higher percentage of regulatory-like B cells might contribute to better clinical outcomes in Tregs+RTX group. Interestingly, it was reported in healthy individuals that serum IgG2 levels were negatively correlated with whole-body insulin sensitivity and muscle insulin sensitivity and associated with insulin-stimulated glucose disposal considering factors known to alter insulin sensitivity like age, gender, BMI, amongst others. (33) changing levels of antibodies after B cell depletion with rituximab also affects infectious immunity. For example, after the flu vaccine in RA patients, humoral response decreased in IgM, IgG1, and IgG3 levels. The phenomenon was time-dependent, observed only in the individuals depleted around one-month pre-vaccination and abolished in individuals who were rituximab treated six to ten months earlier. (34) Although infections were noted in around 60% of the Tregs+RTX cohort in the study according to the present Example, it was comparable with Tregs and control groups. (7)

In summary, the efficacy of the combined therapy can be attributed to several factors. It was mainly associated with an increasing percentage of PD-1+ T cells (on Teff and CD8+ T cells in vivo and Treg in vivo and in vitro) and rebuilding of B cell compartment toward tolerogenic phenotype. The expression of PD-1 on T cells may be a promising biomarker of the efficacy of this therapy. Our data gives a solid background for future clinical trial immune monitoring and sheds light on the immunopathogenesis of DM1.

### REFERENCES

(1) Budd M A, Monajemi M, Colpitts S J, Crome S Q, Verchere C B, Levings M K. Interactions between islets and regulatory immune cells in health and type 1 diabetes. Diabetologia. 2021; 64(11):2378-2388. doi:10.1007/S00125-021-05565-6
(2) Raugh A, Allard D, Bettini M. Nature vs. nurture: FOXP3, genetics, and tissue environment shape Treg function. Front Immunol. 2022; 13:4199. doi:10.3389/FIMMU.2022.911151/BIBTEX
(3) Roep B O. The need and benefit of immune monitoring to define patient and disease heterogeneity, mechanisms of therapeutic action and efficacy of intervention therapy for precision medicine in type 1 diabetes. Front Immunol. 2023; 14. doi:10.3389/FIMMU.2023.1112858
(4) Roep B O, Montero E, van Tienhoven R, Atkinson M A, Schatz D A, Mathieu C. Defining a cure for type 1 diabetes: a call to action. lancet Diabetes Endocrinol. 2021; 9(9):553-555. doi:10.1016/S2213-8587(21)00181-9
(5) Herold K C, Bundy B N, Long S A, et al. An Anti-CD3 Antibody, Teplizumab, in Relatives at Risk for Type 1 Diabetes. N Engl J Med. 2019; 381(7):603-613. doi:10.1056/NEJMOA1902226
(6) Dong S, Hiam-Galvez K J, Mowery C T, et al. The effect of low-dose IL-2 and Treg adoptive cell therapy in patients with type 1 diabetes. JCI insight. 2021; 6(18). doi:10.1172/JCI.INSIGHT.147474
(7) Zieliński M, Żalińska M, Iwaszkiewicz-Grześ D, et al. Combined therapy with CD4+CD25highCD127− T regulatory cells and anti-CD20 antibody in recent-onset type 1 diabetes is superior to monotherapy: Randomised phase I/II trial. Diabetes, Obes Metab. 2022; 24(8):1534-1543. doi:10.1111/DOM.14723
(8) Bluestone J A, Buckner J H, Fitch M, et al. Type 1 diabetes immunotherapy using polyclonal regulatory T cells. Sci Transl Med. 2015; 7(315):315ra189. doi:10.1126/scitranslmed.aad4134
(9) Marek-Trzonkowska N, Myśliwiec M, Dobyszuk A, et al. Therapy of type 1 diabetes with CD4(+)CD25(high)CD127-regulatory T cells prolongs survival of pancreatic islets—results of one year follow-up. Clin Immunol. 2014; 153(1):23-30. doi:10.1016/j.clim.2014.03.016
(10) Marek-Trzonkowska N, Wujtewicz M A, Myśliwiec M, et al. Administration of CD4+CD25highCD127− regulatory T cells preserves β-cell function in type 1 diabetes in children. Diabetes Care. 2012; 35(9):1817-1820. doi:10.2337/DC12-0038
(11) Trzonkowski P, Bacchetta R, Battaglia M, et al. Hurdles in therapy with regulatory T cells. Sci Transl Med. 2015; 7(304). doi:10.1126/SCITRANSLMED.AAA7721
(12) Primary Immunodeficiency Diseases—Immunoglobulin Disorders|Choose the Right Test. https://arupconsult.com/content/immunoglobulin-disorders?_ga=2.163356788.958945285.1684150934-1231057015.1684150933&_gl=1*tlimzr*_ga*MTIzMTA1NzAxNS4xNjg0MTUwOTMz*_ga_Z8H49DQE4D*MTY4NDE1MDkzMy4xLjEuMTY4NDE1MTI0My4wLjAuMA. Accessed May 15, 2023.
(13) Vecchione A, Di Fonte R, Gerosa J, et al. Reduced PD-1 expression on circulating follicular and conventional FOXP3+ Treg cells in children with new onset type 1 diabetes and autoantibody-positive at-risk children. Clin Immunol. 2020; 211. doi:10.1016/J.CLIM.2019.108319
(14) Tucker C G, Dwyer A J, Fife B T, Martinov T. The role of programmed death-1 in type 1 diabetes. Curr Diab Rep. 2021; 21(6):20. doi:10.1007/S11892-021-01384-6
(15) Bluestone J A, Anderson M, Herold K C, et al. Collateral Damage: Insulin-Dependent Diabetes Induced With Checkpoint Inhibitors. Diabetes. 2018; 67(8):1471-1480. doi:10.2337/DBI18-0002
(16) Postow M A, Sidlow R, Hellmann M D. Immune-Related Adverse Events Associated with Immune Checkpoint Blockade. N Engl J Med. 2018; 378(2):158-168. doi:10.1056/NEJMRA1703481
(17) Keir M E, Liang S C, Guleria I, et al. Tissue expression of PD-L1 mediates peripheral T cell tolerance. J Exp Med. 2006; 203(4):883-895. doi:10.1084/JEM.20051776
(18) Guleria I, Gubbels Bupp M, Dada S, et al. Mechanisms of PDL1-mediated regulation of autoimmune diabetes. Clin Immunol. 2007; 125(1):16-25. doi:10.1016/J.CLIM.2007.05.013
(19) Faustini F, Sippl N, Stålesen R, et al. Rituximab in Systemic Lupus Erythematosus: Transient Effects on Autoimmunity Associated Lymphocyte Phenotypes and Implications for Immunogenicity. Front Immunol. 2022; 13:1494. doi:10.3389/FIMMU.2022.826152/BIBTEX
(20) Stasi R, Del Poeta G, Stipa E, et al. Response to B-cell-depleting therapy with rituximab reverts the abnormalities of T-cell subsets in patients with idiopathic thrombocytopenic purpura. Blood. 2007; 110(8):2924-2930. doi:10.1182/BLOOD-2007-02-068999
(21) Stasi R, Cooper N, Poeta G Del, et al. Analysis of regulatory T-cell changes in patients with idiopathic thrombocytopenic purpura receiving B cell-depleting therapy with rituximab. Blood. 2008; 112(4):1147-1150. doi:10.1182/BLOOD-2007-12-129262
(22) Cooper N, Arnold D M. The effect of rituximab on humoral and cell mediated immunity and infection in the treatment of autoimmune diseases. Br J Haematol. 2010; 149(1):3-13. doi:10.1111/J.1365-2141.2010.08076.X
(23) Cooper N, Stasi R, Cunningham-Rundles S, et al. The efficacy and safety of B-cell depletion with anti-CD20 monoclonal antibody in adults with chronic immune thrombocytopenic purpura. Br J Haematol. 2004; 125(2):232-239. doi:10.1111/J.1365-2141.2004.04889.X
(24) Edwards J C W, Szczepański L, Szechiński J, et al. Efficacy of B-cell-targeted therapy with rituximab in patients with rheumatoid arthritis. N Engl J Med. 2004; 350(25):2572-2581. doi:10.1056/NEJMOA032534
(25) Wehr P, Purvis H, Law S C, Thomas R. Dendritic cells, T cells and their interaction in rheumatoid arthritis. Clin Exp Immunol. 2019; 196(1):12-27. doi:10.1111/CEI.13256
(26) Bouaziz J D, Yanaba K, Venturi G M, et al. Therapeutic B cell depletion impairs adaptive and autoreactive CD4+ T cell activation in mice. Proc Natl Acad Sci USA. 2007; 104(52):20878-20883. doi:10.1073/PNAS.0709205105
(27) Pescovitz M D, Greenbaum C J, Krause-Steinrauf H, et al. Rituximab, B-Lymphocyte Depletion, and Preservation of Beta-Cell Function. N Engl J Med. 2009; 361(22):2143-2152. doi:10.1056/NEJMOA0904452/SUPPL_FILE/NEJM_PESCOVITZ_2143SA1.PDF
(28) Pescovitz M D, Greenbaum C J, Bundy B, et al. B-Lymphocyte Depletion With Rituximab and β-Cell Function: Two-Year Results. Diabetes Care. 2014; 37(2):453-459. doi:10.2337/DC13-0626
(29) Kavuru M S, Malur A, Marshall I, et al. An open-label trial of rituximab therapy in pulmonary alveolar proteinosis. Eur Respir J. 2011; 38(6):1361-1367. doi:10.1183/09031936.00197710
(30) Khosroshahi A, Carruthers M N, Deshpande V, Unizony S, Bloch D B, Stone J H. Rituximab for the treatment of IgG4-related disease: lessons from 10 consecutive patients. Medicine (Baltimore). 2012; 91(1):57-66. doi:10.1097/MD.0B013E3182431EF6
(31) Avery D T, Bryant V L, Ma C S, de Waal Malefyt R, Tangye S G. IL-21-induced isotype switching to IgG and IgA by human naive B cells is differentially regulated by IL-4. J Immunol. 2008; 181(3):1767-1779. http://www.ncbi.nlm.nih.gov/pubmed/18641314. Accessed Nov. 15, 2018.
(32) Hamilton R, Abmli D, Mohan C. The Human IgG Subclasses. Hum IgG Subclasses. 2001. doi:10.1016/C2009-0-00373-0
(33) Fiorentino T V, Succurro E, Arturi F, et al. Serum IgG2 levels are specifically associated with whole-body insulin-mediated glucose disposal in non-diabetic offspring of type 2 diabetic individuals: a cross-sectional study. Sci Rep. 2018; 8(1). doi:10.1038/S41598-018-32108-8
(34) Westra J, Van Assen S, Wilting K R, et al. Rituximab impairs immunoglobulin (Ig)M and IgG (subclass) responses after influenza vaccination in rheumatoid arthritis patients. Clin Exp Immunol. 2014; 178(1):40-47. doi:10.1111/CEI.12390
(35) Marek N, Bieniaszewska M, Krzystyniak A, et al. The time is crucial for ex vivo expansion of T regulatory cells for therapy. Cell Transpl. 2011; 20(11-12):1747-1758. doi:10.3727/096368911X566217
(36) Marek-Trzonkowska N, Piekarska K, Filipowicz N, et al. Mild hypothermia provides Treg stability. Sci Rep. 2017; 7(1):11915. doi:10.1038/s41598-017-10151-1
(37) Belkina A C, Ciccolella C O, Anno R, Halpert R, Spidlen J, Snyder-Cappione J E. Automated optimised parameters for T-distributed stochastic neighbor embedding improve visualisation and analysis of large datasets. Nat Commun. 2019; 10(1). doi:10.1038/S41467-019-13055-Y
(38) Amid E, Warmuth M K. TriMap: Large-scale Dimensionality Reduction Using Triplets. December 2019
(39) Levine J H, Simonds E F, Bendall S C, et al. Data-Driven Phenotypic Dissection of AML Reveals Progenitor-like Cells that Correlate with Prognosis. Cell. 2015; 162(1):184-197. doi:10.1016/j.cell.2015.05.047
(40) Babicki S, Arndt D, Marcu A, et al. Heatmapper: web-enabled heat mapping for all. Nucleic Acids Res. 2016; 44(W1):W147-53. doi:10.1093/nar/gkw419
(41) Zieliński M, Żalińska M, Iwaszkiewicz-Grześ D, et al. Combined therapy with CD4+CD25highCD127− T regulatory cells and ant-CD20 antibody in recent-onset type 1 diabetes is superior to monotherapy: Randomized phase I/II trial. Diabetes, Obes Metab. 2022; 24(8):1534-1543. doi:10.1111/DOM.14723
(42) Faber O K, Binder C. C-peptide response to glucagon. A test for the residual beta-cell function in diabetes mellitus. Diabetes. 1977 (26): 605-610. doi: 10.2337/diab.26.7.605.

### Tables

TABLE 1A
Clinical trial participant's patient's baseline characteristics

| | Treg + | Treg + | | |
|---|---|---|---|---|
| | placebo | rituximab | | |
| | [Treg] | [Treg + RTX] | Control | |
| | (n = 13) | (n = 12) | (n = 11) | |
| Characteristic | mean ± SD | mean ± SD | mean ± SD | p-value |
| Male sex, n (%) | 7 (52.8) | 5 (41.7%) | 5 (45.5) | 0.61 |
| Age, years | 13.3 ± 1.5 | 12.9 ± 1.2 | 12.1 ± 2.2 | 0.68 |
| Body mass index, kg/m² | 19.57 ± 1.8 | 18.1 ± 1.8 | 18.4 ± 1.4 | 0.45 |
| Body mass index, Z-core | −0.24 ± 0.46 | −0.01 ± 0.43 | 0.04 ± 0.42 | 0.19 |
| Ethnicity, Caucasian (%) | 13 (100.0) | 12 (100.0) | 11 (100.0) | — |
| Months since diagnosis | 6.5 ± 4.2 | 6.0 ± 4.2 | 5.0 ± 3.2 | 0.48 |
| Insulin (TDD per kg of | 0.3 ± 0.3 | 0.2 ± 0.2 | 0.3 ± 0.3 | 0.71 |
| body weight) | | | | |
| C-peptide | | | | |
| Fasting C-peptide | 1.1 ± 0.4 | 1.1 ± 0.3 | 0.98 ± 0.2 | 0.45 |
| (μg/L) | | | | |
| Stimulated C-peptide | 10.1 ± 2.4 | 11.0 ± 3.7 | 9.8 ± 2.1 | 0.38 |
| AUC240 (h*μg/L) | | | | |
| Glycated haemoglobin | 6.3 ± 1.1 | 6.6 ± 1.2 | 6.6 ± 0.8 | 0.72 |
| (%) | | | | |
| Glucose (mg/dl) | 103.1 ± 9.7 | 109.5 ± 13.5 | 103.9 ± 13.3 | 0.55 |
| (mean from fasting from | | | | |
| seven days before the | | | | |
| visit) | | | | |
| Autoantibodies | | | | |
| Glutamic acid | 856.6 ± 936.5 | 381.0 ± 594.6 | 744.2 ± 768.1 | 0.74 |
| decarboxylase (IU/ml) | | | | |
| Insulin autoantibody | 8.7 ± 8.6 | 5.3 ± 5.5 | 5.0 ± 5.1 | 0.38 |
| (IU/ml) | | | | |
| Islet cell antibody (titre) | 109.2 ± 180.5 | 125.0 ± 185.0 | 50.9 ± 47.6 | 0.25 |

P-values in Table 1A are based on one-way ANOVA F-statistics for continuous and Kruskal-Wallis statistics for multilevel categorical data; adopted from (7).

TABLE 1B
DM1 in-vitro model patient's baseline characteristics

| | Type 1 | | |
|---|---|---|---|
| | diabetes | Healthy | |
| | patients | controls | |
| | [DM1] | [HC] | |
| | (n = 12) | (n = 12) | |
| Characteristic | mean ± SD | mean ± SD | p-value |
| Male sex, n (%) | 9 (75) | 10 (83.3) | >0.99 |
| Age, years | 11.6 ± 2.8 | 29.8 ± 6.1 | <0.0001 |
| Ethnicity, Caucasian (%) | 12 (100.0) | 12 (100.0) | not applicable |

The p-values are based on Fisher's exact test for categorical data and the t-test for continuous data. Healthy controls were anonymous blood donors, all characteristics assumed to be within normal range. n/a—not applicable

TABLE 2
Clinical trial and in-vitro model participant's inclusion and exclusion criteria

| Inclusion criteria | Exclusion criteria |
|---|---|
| 1. Participant age: 8-16 years of age | 1. No agreement for participation in the |
| | study and no informed consent signed. |
| 2. BMI: 25th-75th percentile (according to | 2. Other than autoimmune type 1 diabetes. |
| the OLAF project) | |
| 3. Fasting plasma C-peptide >0.7 ng/ml and | 3. Age <8 and >16 years. |
| in stimulation test the increase ≥100% | |
| 4. The presence of at least one anti-islet | 4. IgA deficiency or other genetic defects |
| autoantibody (ICA, IAA, GAD): a high titre of | present. |
| IAA or GAD (>4 times the norm) or a low | |
| titre (2-4 times the norm) of at least two of | |
| these antibodies. | |
| 5. Ability to provide written informed | 5. BMI <25th or >75th percentile for a |
| consent by parents (and patients if >16 | particular age. |
| years old). | |
| 6. Involvement of the patients and parents | 6. Hypersensitivity to anti-CD20 antibody |
| in intensive diabetes management is | rituximab or other components of the |
| defined as self-monitoring of glucose values | preparation. |
| no less than three times per day and | |
| administering insulin. | |
| 7. Appropriate venous access for blood | 7. Presence or history of active infection, |
| drawing. | including hepatitis B, hepatitis C, HIV, |
| | tuberculosis or syphilis. Subjects with |
| | laboratory evidence of active infection were |
| | excluded even without clinical evidence of |
| | active infection. |
| | 8. Presence of active EBV virus infection |
| | (positive IgM). |
| | 9. Presence or history of active systemic |
| | fungal infection. |
| | 10. Any history of malignancy. |
| | 11. Anaemia, lymphopenia, neutropenia or |
| | thrombocytopenia below the lower limits of |
| | the reference range during the 6 weeks |
| | before the study. |
| | 12. Known hypercoagulative state. |
| | 13. Medical treatment requiring chronic use |
| | of drugs other than insulin longer than 3 |
| | months. |
| | 14. Treatment with anti-diabetic medication |
| | other than insulin within 4 weeks of |
| | enrolment. |
| | 15. Diabetic retinopathy. |
| | 16. Arterial hypertension. |
| | 17. Presence or history of |
| | macroalbuminuria. |
| | 18. For female subjects older than 15 years: |
| | a positive pregnancy test or an |
| | unwillingness to use adequate |
| | contraceptive measures for the study and 4 |
| | months after discontinuation, when |
| | appropriate. |
| | 19. For male subjects: intent to procreate |
| | during the duration of the study or within 4 |
| | months after discontinuation when |
| | appropriate. |
| | 20. Excessive anxiety of the patient or |
| | parents related to the procedures. |
| | 21. Any medical condition that, in the |
| | investigator's opinion, will interfere with safe |
| | participation in the trial. |
| | 22. For parents and paediatric patients |
| | older than 15 years: known active alcohol or |
| | substance abuse. |

Adapted from (41).

TABLE 3A
Flow cytometry staining chart-whole blood lymphocytes T flow cytometry antigen panel

| | Tube 1 | Tube 2 | | | |
|---|---|---|---|---|---|
| | unstained | backbone | | | |
| Fluorochrome | sample | markers | Tube 3 | Tube 4 | Tube 5 |
| FITC | not | BD; CD127 | BD; CD18 | BD; CD127 | BD; CD127 |
| | applicable | clone: HIL- | clone: L130 | clone: HIL- | clone: HIL- |
| | | 7R-M21 | | 7R-M21 | 7R-M21 |
| PerCP | not | BD; CD4 | R&D | BD; CD4 | BD; CD4 |
| | applicable | clone: SK3 | Systems; | clone: SK3 | clone: SK3 |
| | | | CCR8 | | |
| | | | clone: | | |
| | | | 191704 | | |
| APC | not | Invitrogen; | Invitrogen; | Invitrogen; | Invitrogen; |
| | applicable | FoxP3 | FoxP3 | FoxP3 | FoxP3 |
| | | clone: | clone: | clone: | clone: |
| | | PCH101 | PCH101 | PCH101 | PCH101 |
| Alexa Fluor 700 | not | — | BD; CD4 | Invitrogen; | — |
| | applicable | | clone: SK3 | CD304 | |
| | | | | clone: | |
| | | | | TNKUSOHA | |
| Alexa Fluor 780 | not | Invitrogen; | Invitrogen; | Invitrogen; | Invitrogen; |
| | applicable | CD62L | CD62L | CD62L | CD62L |
| | | clone: | clone: | clone: | clone: |
| | | DREG-56 | DREG-56 | DREG-56 | DREG-56 |
| PE | not | BD; CD25 | R&D | BD; CD25 | BD; CD25 |
| | applicable | clone: M- | Systems; | clone: M- | clone: M- |
| | | A251 | CCR10 | A251 | A251 |
| | | | clone: | | |
| | | | 314305 | | |
| PE-Texas Red | not | — | BD; CD184 | — | — |
| | applicable | | clone: 12G5 | | |
| PE-Cy7 | not | BD; | BD; CD45RA | BD; | BD; |
| | applicable | CD45RA | clone: HI30 | CD45RA | CD45RA |
| | | clone: HI30 | | clone: HI30 | clone: HI30 |
| eFluor450 | not | Invitrogen; | Invitrogen; | Invitrogen; | Invitrogen; |
| | applicable | Helios | Helios | Helios | Helios |
| | | clone: 22F6 | clone: 22F6 | clone: 22F6 | clone: 22F6 |
| V500 | not | BD; CD3 | BD; CD3 | BD; CD3 | BD; CD3 |
| | applicable | clone: | clone: | clone: | clone: |
| | | UCHT1 | UCHT1 | UCHT1 | UCHT1 |
| Brilliant Violet 605 | not | — | BD; CD194 | — | BD; CD279 |
| | applicable | | clone: 1G1 | | clone: |
| | | | | | EH12.1 |
| Brilliant Violet 650 | not | — | — | BD; CD39 | BD; CD137 |
| | applicable | | | clone: TU66 | clone: 4B4-1 |
| Brilliant Violet 711 | not | — | — | — | BD; CD134 |
| | applicable | | | | clone: |
| | | | | | ACT35 |
| Brilliant Violet 786 | not | — | BD; CD25 | BD; CD152 | — |
| | applicable | | clone: M- | clone: BNI3 | |
| | | | A251 | | |
| Brilliant Ultra | not | — | BD; CD103 | — | — |
| Violet 395 | applicable | | clone: Ber- | | |
| | | | ACT8 | | |
| Brilliant Ultra | not | — | BD; CD127 | BD; CD73 | BD; CD39 |
| Violet 737 | applicable | | clone: HIL- | clone: AD2 | clone: TU66 |
| | | | 7R-M21 | | |

For the surface/intracellular staining, a protocol was used strictly according to the eEioscience's Foxp3/Transcription Factor Staining Buffer Set, Thermo Fisher Scientific (Waltham, MA, USA).

TABLE 3B
Flow cytometry staining chart-whole blood lymphocytes B
flow cytometry antigen panel

| | Tube 1 | Tube 2 | |
|---|---|---|---|
| | unstained | backbone | Tube 3 |
| Fluorochrome | sample | markers | full stain |
| FITC | not applicable | — | eBioscience; CD5 |
| | | | clone: L17F12 |
| PerCP-eFluor | not applicable | — | eBioscience; CD10 |
| 710 | | | clone: |
| | | | eBioSN5c(SN5cL4- |
| | | | 1A1) |
| APC | not applicable | — | eBioscience; CD21 |
| | | | clone: HB5 |
| APC eFluor | not applicable | — | eBioscience; CD24 |
| 780 | | | clone: |
| | | | eBioSN3(SN3A5- |
| | | | 2H10) |
| PE | not applicable | — | eBioscience; IgD |
| | | | clone: IA6-2 |
| PE-eVolve | not applicable | — | eBioscience; CD38 |
| 655 | | | clone: HIT2 |
| PE-Cy7 | not applicable | BD; CD20 | BD; CD20 |
| | | clone: L27 | clone: L27 |
| eFluor 450 | not applicable | — | eBioscience; IgM |
| | | | clone: SA-DA4 |
| V500 | not applicable | BD; CD19 | BD; CD19 |
| | | clone: HIB 19 | clone: HIB 19 |
| eVolve 655 | not applicable | — | eBioscience; CD27 |
| | | | clone: O323 |

TABLE 3C
Flow cytometry staining chart-In-vitro model lymphocytes
T flow cytometry antigen panel

| | Tube 1 | Tube 2 | |
|---|---|---|---|
| | unstained | backbone | Tube 3 |
| Fluorochrome | sample | markers | full stain |
| FITC | not applicable | Invitrogen; CD3 | Invitrogen; CD3 |
| | | clone: UCHT1 | clone: UCHT1 |
| PerCP | not applicable | BD; CD4 | BD; CD4 |
| | | clone: SK3 | clone: SK3 |
| Brilliant Ultra | not applicable | BD; CD8 | BD; CD8 |
| Violet 395 | | clone: RPA-T8 | clone: RPA-T8 |
| APC | not applicable | | Invitrogen; FoxP3 |
| | | | clone: PCH101 |
| Alexa Fluor 700 | not applicable | | Invitrogen; Helios |
| | | | clone: 22F6 |
| APC-eFluor 780 | not applicable | | Invitrogen; CD62L |
| | | | clone: DREG-56 |
| SuperBright600 | not applicable | | Invitrogen; |
| | | | CD45RA |
| | | | clone: HI100 |
| PE-Cy7 | not applicable | | Invitrogen; CD28 |
| | | | clone: CD28.2 |
| eFluor450 | not applicable | | Invitrogen; CD57 |
| | | | clone: TB01 |
| SuperBright702 | not applicable | | Invitrogen; CD279 |
| | | | clone: eBioJ105 |
| | | | (J105) |

For the surface/intracellular staining, a protocol was used strictly according to the eBioscience's Foxp3/Transcription Factor Staining Buffer Set, Thermo Fisher Scientific (Waltham, MA, USA).

TABLE 4
Area under the ROC curve for the selected parameter

| | | | | |
|---|---|---|---|---|
| | the area | | | |
| | under | the | 95% | |
| | the ROC | standard | confidence | |
| | curve | error | interval | |
| Cell phenotype | (AUC) | (SE) | (95% CI) | p-value |

TREG + RTX

| | | | | |
|---|---|---|---|---|
| PD1 + Treg [%] | 0.740 | 0.126 | 0.5041-0.9959 | 0.041 (*) |
| PD1 + Teff [%] | 0.750 | 0.114 | 0.5266-0.9734 | 0.042 (*) |
| PD1 + CD8 [%] | 0.632 | 0.116 | 0.4038-0.8600 | 0.272 (NS) |
| CD73 + Treg [%] | 0.849 | 0.082 | 0.6888-1.000 | 0.005 ( ** ) |
| CD73 + Teff [%] | 0.535 | 0.122 | 0.2948-0.7746 | 0.773 (NS) |
| CD39 + Treg [%] | 0.583 | 0.121 | 0.3465-0.8201 | 0.488 (NS) |
| CD39 + Teff [%] | 0.597 | 0.120 | 0.3630-0.8315 | 0.419 (NS) |
| CD304 + Helios + | 0.611 | 0.119 | 0.3785-0.8437 | 0.356 (NS) |
| Treg [%] | | | | |
| IgG1 [mg/ml] | 0.592 | 0.125 | 0.3465-0.8368 | 0.468 (NS) |
| IgG2 [mg/ml] | 0.692 | 0.118 | 0.4597-0.9236 | 0.129 (NS) |

TREG

| | | | | |
|---|---|---|---|---|
| PD1 + Treg [%] | 0.530 | 0.133 | 0.2696-0.7903 | 0.815 (NS) |
| PD1 + Teff [%] | 0.513 | 0.127 | 0.2634-0.7623 | 0.920 (NS) |
| PD1 + CD8 [%] | 0.704 | 0.128 | 0.4531-0.9544 | 0.118 (NS) |
| CD73 + Treg [%] | 0.660 | 0.110 | 0.4438-0.8762 | 0.183 (NS) |
| CD73 + Teff [%] | 0.608 | 0.122 | 0.3688-0.8466 | 0.385 (NS) |
| CD39 + Treg [%] | 0.508 | 0.132 | 0.2499-0.7655 | 0.951 (NS) |
| CD39 + Teff [%] | 0.600 | 0.124 | 0.3570-0.8430 | 0.420 (NS) |
| CD304 + Helios + | 0.512 | 0.126 | 0.2647-0.7584 | 0.926 (NS) |
| Treg [%] | | | | |
| IgG1 [mg/ml] | 0.688 | 0.128 | 0.4374-0.9393 | 0.189 (NS) |
| IgG2 [mg/ml] | 0.701 | 0132 | 0.4421-0.9605 | 0.160 (NS) |

1-29. (canceled)

30. A method for monitoring a cell therapy of a patient treated with CD4+ Fox P3+ T regulatory cells comprising the step(s) of determining the expression of at least one protein selected from the group consisting of PD-1 and CD73 on CD4+ FoxP3+ T cells and/or determining the expression of PD-1 on CD4+ FoxP3− cells of the patient in vitro.

31. The method of claim 30, wherein determining the expression of the at least one protein on CD4+ FoxP3+ T cells and/or determining the expression PD-1 on CD4+ FoxP3− cells is carried out on a sample of the patient.

32. The method of claim 30, wherein the method comprises, before determining the expression of the at least one protein, isolating CD4+ FoxP3+ T cells from a sample of the patient and/or, before determining the expression of PCD1 on CD4+ FoxP3− cells, isolating CD4+ FoxP3− cells from the sample of the patient.

33. The method of claim, 32 further comprising expanding the isolated CD4+ FoxP3+ T cells and/or the isolated CD4+ FoxP3− cells.

34. The method of claim 31, wherein the sample is peripheral blood.

35. The method of claim 33, wherein the isolated and expanded CD4+ FoxP3+ T cells are CD4+ FoxP3+ T regulatory cells.

36. The method of claim 33, wherein the isolated and expanded CD4+ FoxP3− T cells are CD4+ FoxP3− T effector cells.

37. The method of claim 30, comprising the steps of:
(i) determining the expression of PD-1 on the CD4+ FoxP3+ T regulatory cells; and/or
(ii) determining the expression of PD-1 on the CD4+ FoxP3− T effector cells; and/or
(iii) determining the expression of CD73 on the CD4+ FoxP3+ T regulatory cells.

38. The method of claim 35, wherein the T regulatory cells are of the phenotype CD4+ FoxP3+ CD25high CD127− doublets−.

39. The method of claim 36, wherein the T effector cells are of the phenotype CD4+ FoxP3− CD25low CD127+ doublets−.

40. The method of claim 30, wherein the method is carried out on the cells of the patient one or more times at least 2 weeks, after the administration of the CD4+ Fox P3+ T regulatory cells.

41. The method of claim 40, wherein the method is carried out on the cells of the patient in intervals of at least at least 2 weeks after the administration of the CD4+ Fox P3+ T regulatory cells.

42. The method of claim 40, wherein the method is carried out on the cells of the patient for at least 6 months after the administration of CD4+ Fox P3+ T regulatory cells.

43. The method of claim 40, wherein the method is carried out at least once on the cells of the patient prior to the administration of the CD4+ Fox P3+ T regulatory cells.

44. The method of claim 30, wherein the patient has an autoimmune disease.

45. The method of claim 44 wherein the autoimmune disease is type 1 diabetes mellitus.

46. The method of claim 45 wherein the patient is a child or an adolescent.

47. The method of claim 30, wherein the patient is further treated with an anti-CD20 antibody or fragment thereof retaining specific binding to CD20.

48. The method of claim 47, wherein the anti-CD20 antibody is rituximab.

49. A method for the assessment of the efficacy of a cell therapy of a patient treated with CD4+ Fox P3+ T regulatory cells comprising carrying out the method of claim 30, wherein the expression of PD-1 on at least 16% of the CD4+ FoxP3+ T regulatory cells and/or the expression of CD73 on at least 7% of the CD4+ FoxP3+ T regulatory cells and/or the expression of PD-1 on at least 8% of the CD4+ FoxP3− T cells indicates an efficient cell therapy.

50. The method of claim 30, further comprising administering CD4+ Fox P3+ T regulatory cells to a patient having or developing an autoimmune disease.

51. The method of claim 50, further comprising administration of an anti-CD20 antibody or fragment thereof retaining specific binding to CD20.

52. The method of claim 50, wherein the anti-CD20 antibody is rituximab.

53. The method of claim 50, wherein the autoimmune disease is type 1 diabetes mellitus.

54. The method of claim 53, wherein the patient is a child or an adolescent.

55. A method of diagnosing an efficient cell therapy of a patient treated with CD4+ Fox P3+ T regulatory cells comprising the steps of obtaining a sample of a patient, isolating CD4+ FoxP3+ T cells and/or CD4+ FoxP3− cells from the patient, contacting the isolated CD4+ FoxP3+ T cells and/or isolated CD4+ FoxP3− cells with a detectable compound having high affinity for a protein selected from the group consisting of PD-1 and CD73, detecting said detectable compound bound to CD4+ FoxP3+ T cells expressing PD-1 and/or CD73 and/or bound to CD4+ FoxP3− cells expressing PD-1, and determining the proportion of the CD4+ FoxP3+ T cells expressing PD-1 and/or CD73 and/or the proportion of CD4+ FoxP3− cells expressing PD-1.

56. The method of claim 55, wherein the isolated CD4+ FoxP3+ T cells and/or isolated CD4+ FoxP3− cells are expanded before contacting said cells with said detectable compound.

57. The method of claim 55, wherein the detectable compound is an anti-PD1-antibody or fragment thereof retaining specific binding to PD-1 or an anti-CD73-antibody or fragment thereof retaining specific binding to CD73.

58. The method of claim 55, wherein the compound is coupled to a detectable label.

59. The method of claim 58, wherein the label is a fluorescent label.
